(19) United States
(12) Patent Application Publication
(10) Pub. No.: US 2022/0204663 A1
(43) Pub. Date: Jun. 30, 2022

(54) BIO-BASED ETHYLENE FOR THE PRODUCTION OF BIO-BASED POLYMERS, COPOLYMERS, AND OTHER BIO-BASED CHEMICAL COMPOUNDS

(71) Applicant: Xyleco, Inc., Wakefield, MA (US)

(72) Inventors: Mohanreddy Kasireddy, North Andover, MA (US); Balaraju Miryala, North Andover, MA (US); Marshall Medoff, Brookline, MA (US); Thomas Craig Masterman, Rockport, MA (US); Christopher G. Cooper, Rehoboth, MA (US)

(21) Appl. No.: 17/606,984

(22) PCT Filed: Apr. 29, 2020
(86) PCT No.: PCT/US2020/030451

§ 371 Date: Oct. 27, 2021

Related U.S. Application Data
(60) Provisional application No. 62/947,341, filed on Dec. 12, 2019.
(60) Provisional application No. 62/936,096, filed on Nov. 15, 2019.
(60) Provisional application No. 62/897,002, filed on Sep. 6, 2019.
(60) Provisional application No. 62/840,903, filed on Apr. 30, 2019.

(51) Int. Cl. C08F 110/02

(52) U.S. Cl. CPC C08F 110/02

(57) ABSTRACT

Bio-based ethanol, such as ethanol produced from lignocellulosic materials, for example, is processed to produce bio-based ethylene, which can then be processed further to produce other bio-based materials including bio-based polymers and copolymers, including bio-based polyethylene, bio-based α-olefins, bio-based 1,2-diols, as well as other compounds.

This application claims priority to U.S. Provisional Application 62/840,903, filed on Apr. 30, 2019, U.S. Provisional Application 62/897,002, filed on Sep. 6, 2019, U.S. Provisional Application 62/936,096, filed on Nov. 15, 2019, and U.S. Provisional Application 62/947,341, filed on Dec. 12, 2019, all of which are hereby incorporated by reference in their entireties.

### FIELD OF THE INVENTION

The present invention relates to materials and methods for producing bio-based ethylene, which can be used to make other bio-based products such as, e.g., bio-based polyethylene; other bio-based polymers; copolymers; α-olefins containing four, six, eight, ten or more carbon atoms; 1,2-diols; and many other bio-based chemical compounds.

### BACKGROUND OF THE INVENTION

Polyethylene is an inexpensive and versatile material that is utilized extensively for many different applications. Most polyethylene is made from ethylene produced from oil or from natural gas liquids. In the U.S., most ethylene is produced from extracting ethane from natural gas liquids and cracking the ethane to ethylene. Regardless of its source (oil or natural gas liquids), such ethylene is made from a limited, non-renewable and ancient hydrocarbon resource, and its production releases carbon that was stored within the earth.

Methods of sustainably producing ethylene that do not rely on oil and natural gas as the source material would reduce consumption of fossil fuels and would preserve this limited, non-renewable resource for the production of materials that provide for the survival of humankind, such as for the production of live-saving drugs.

### SUMMARY OF THE INVENTION

Ethylene and other compounds produced or derived from ethylene from biomass, such as lignocellulosic biomass, such as naturally-derived preservatives, such as 1,2-diols, may be utilized in a variety of consumer goods, such as cosmetics in which natural, naturally derived, natural-origin, high modern carbon content, or bio-based materials, such as 100 percent natural-origin materials, are in high demand and often command a price premium as consumers seek natural alternatives to petroleum-based materials. The present invention provides compositions and methods of sustainably producing such compounds that would reduce the consumption of limited and non-renewable fossil fuels, while drastically reducing or practically eliminating the introduction of new atmospheric carbon in comparison to conventional synthetic processes.

The present invention provides such compositions and methods, at least in part, by providing for the sustainable production of bio-based ethylene, which can be made from ethanol produced from biomass, such as from a cellulosic or lignocellulosic material, e.g., corn stover or corn cobs, a starchy material, e.g., corn grain or cassava, or a sucrose-rich source, such as sugarcane or sugar beets. Bio-based ethylene can also be used to make a large variety of bio-based chemical compounds, such as α-olefins containing four, six, eight, ten or more carbons atoms, e.g., 12, 14, 16, 18, 20, 22, 24, or more carbons atoms, such as 26 or more carbon atoms. The bio-based α-olefins can then be used in a wide variety of chemical pathways to subsequently sustainably produce many other possible compounds, such as for example, phenoxyethanol, a haloethanol, such as a chloroethanol, such as 2-chloroethanol, 1,2-diols (in any stereoisomeric form, R-, S- or R/S-), and specifically 1,2-octanediol (also known as caprylyl glycol or octane-1,2-diol in any stereoisomeric form), as described herein. Other uses of alpha olefins include as polyethylene co-monomers, and as building blocks for surfactants, plasticizers, synthetic motor oils, synthetic lubricants, synthetic cutting and drilling fluids and additives for lubricating oils. Depending upon the method used herein, such resulting compounds can be 100 percent modern carbon bio-based materials or derived natural ingredients, such as those that are 100 percent natural origin.

Bio-based ethylene may also be utilized to synthesize bio-based vinyl acetate monomers, bio-based ethylene-vinyl acetate (EVA) copolymers, and bio-based EVA foams as described herein. For example, as further described herein, a vinyl acetate monomer can be produced from the reaction of bio-based ethylene or mixtures with bio-based ethylene and fossil ethylene when less than 100 percent modern carbon monomer is desired, for example, due to costing, and acetic acid in the presence of a catalyst, for example, a palladium catalyst. The resulting vinyl acetate polymers, e.g., vinyl acetate homopolymer or copolymer, can be mixed with other polymers, e.g., intimately melt blended, to produce polymer blends of other bio-based polymers or fossil fuel based polymers to produce blends of varying amounts of modern carbon content.

The use of fossil fuels as an energy or fuel source, such as a source of heat or electricity, or as a source of raw materials for certain compounds described herein, results in the generation of greenhouse gases, such as carbon dioxide or stray hydrocarbons formed during processing, such as escaped natural gases including methane, which have the tendency to trap the sun's radiated heat. Global warming potential (GWP) is an index measurement of an emitted substance's radiative forcing, or how much of the sun's heat that substance traps, compared to an equivalent mass of CO₂. Blends of fossil fuel-derived ethylene and biomass-derived ethylene, or a blend of any biomass-derived material, such as any monomeric or polymeric material described herein, and its fossil fuel-derived equivalent, such as blends of fossil fuel derived caprylyl glycol and biomass derived caprylyl glycol, can be utilized if desired, for example, as a method of providing a favorable balance between cost and GWP reduction. For example, a 70 percent modern carbon content material, such as caprylyl glycol or phenoxyethanol, can be provided by mixing 70 percent by weight of a bio-based, high modern carbon content material with 30 percent by weight of its fossil fuel-derived equivalent. In other instances, any one or more biomass-derived materials described herein can be diluted with any one or more fossil fuel-derived materials to produce a desired percent modern carbon or natural origin content, such as greater than 25 percent, such as greater than 35, 45, 50, 55 percent, such as greater than about 60 percent, such as greater than about 65, 70, 75, 80, 85, 95 percent or more, such as greater than about 96, 97, 98 percent or more, such as greater than about 99.5 percent, or even about 100 percent. In particular instances in which the ethylene is produced from biomass-derived ethanol, the ethanol can be produced from a cellulosic source, a lignocellulosic source, a starchy source, such as from corn grain, or a source including a low molecular weight carbohydrate, such as sugarcane or sugar beets. Such a strategy can be useful at reducing further emissions of carbon dioxide to the earth's atmosphere, which have increased from around 316 ppm in 1958 to about 413 ppm today, representing over a thirty-percent increase in atmospheric carbon dioxide levels since 1958.

For any product described herein derived from biomass, for example, ethylene or 1,2-octanediol (in any isomeric form or isomeric blend, e.g., racemic blend), the product can have, for example, a global warming potential reduction of between about 5 and about 90 percent, or between about 10 and about 85 percent, between about 15 and about 75 percent, between about 20 and about 65, or between about 22 and about 55 percent reduction relative to its fossil fuel-derived counterpart. In other embodiments, the GWP reduction can be greater than about 5, 10, 15, 20, 25, 30, 35 percent or more, e.g., greater than about 50, 60, 70 percent or more, such as greater than about an 80 percent reduction relative to its fossil fuel-derived counterpart.

In particular embodiments, in which the product is derived from a lignocellulosic feedstock or a sucrose rich feedstock, such as sugarcane or sugar beets, the GWP reduction can be greater than about 70, 72, 74, 76 percent, or more, e.g., greater than about 80, 81, 82, 83, 84, or even greater than about 85 percent reduction relative to its fossil fuel-derived counterpart.

Any one or more monomeric or polymeric product described herein derived from biomass or a combination of one or more biomass-derived products and a fossil fuel resource, can have, for example, in addition to the GWP reduction described above, a percent modern carbon (pMC) of greater than 10 percent or more, such as greater than about 15, 20, 25, 30, 35, 40, or more, e.g., greater than about 50, 60, 70, 80, 90 or more pMC, such as greater than about 95 pMC. In particular instances, the pMC is greater than even about 95 pMC, such as greater than about 96, 97, 98.5, 99, 99.5 or more, e.g., greater than about 99.8, 99.9 or more pMC. In particular instances, it is essentially about 100 pMC.

High modern-carbon content molecules and mixtures are generally provided by the invention described herein. This invention produces carbon-based molecules and mixtures of high natural-origin or modern carbon content suitable for making items utilized worldwide by billions of people daily including plastics, e.g., degradable plastics, e.g., polyvinyl alcohol, pharmaceuticals, building materials, agricultural materials, and consumer goods, such as cosmetics and footwear. High modern-carbon content molecules reduce carbon dioxide emissions into the environment and are based on sustainable life, e.g., plant life, such as terrestrial or water-based plant life (e.g., aquatic), e.g., marine plant life, including carbohydrate rich materials such as starchy materials, cellulosic materials, and lignocellulosic materials.

The present invention provides, in part, materials and methods for processing bio-based ethanol, such as lignocellulosic ethanol or ethanol from starchy materials or sucrose rich materials, to produce bio-based ethylene and/or carbon-containing materials derived therefrom utilizing one or more chemical reactions. In certain embodiments, the ethanol is produced from biomass, such as lignocellulosic biomass, which includes, for example, corn stover, corn cobs, and wheat straw. The ethylene produced from such biomass-derived ethanol, such as from lignocellulosic ethanol or ethanol from a starchy material, is referred to herein as bio-based ethylene. Bio-based ethylene, like fossil fuel-based ethylene, can be used to produce a variety of chemical products, including polymers and copolymers. For example, the bio-based ethylene can be polymerized to bio-based polyethylene, including low, medium, or high density bio-based polyethylene. Such bio-based polyethylene is made of modern carbon having a low carbon footprint. In addition to polymerizing bio-based ethylene, such bio-based ethylene can be used in various oxidation reactions (e.g., to produce polyethylene oxide), halogenation reactions (e.g., to produce vinyl chloride, perchloroethylene, or vinylidene dichloride), alkylation reactions, hydration reactions, and hydroformylation reactions. In such instances, a fraction, e.g., greater than about 30, 40, 50, 60 or even greater than 75 percent, of the carbon in the resulting chemicals would be modern carbon. Bio-based ethylene can also be used to make a large variety of bio-based chemical compounds, such as α-olefins containing four, six, eight, ten or more carbons. The bio-based α-olefins or bio-based ethylene can then be used in a wide variety of chemical pathways to subsequently produce many possible compounds, such as for example, phenoxyethanol, 1,2-diols, and specifically 1,2-octanediol, also called caprylyl glycol, as described herein. The bio-based ethylene may also be used to make bio-based vinyl acetate monomers, bio-based ethylene-vinyl acetate (EVA) copolymers, and bio-based EVA foams as described herein. While bio-based ethylene is an important building block as described herein, it can be powerfully augmented with other bio-based transformations described herein. For example, utilizing bio-based ethylene, important bio-based intermediates can be produced, for example, bio-based ethylene oxide or bio-based 2-chloroethanol. Other bio-based transformations can be utilized to provide yet other bio-based intermediates. For example, bio-based guaiacol can be catalytically de-methoxylated to produce bio-based methanol and bio-based phenol. The resulting bio-based phenol can be reacted with the 2-chloroethanol to produce bio-based phenoxyethanol, which can be utilized as a preservative, for example, in cosmetic formulations. The bio-based methanol produced from the catalytic de-methoxylation reaction can be utilized, for example, to produce methyl esters in other reactions to produce high modern carbon content methyl esters. In one implementation, guaiacol can be produced by pyrolysis (in the absence of air) of woody materials, such as pine materials, such as those materials found toward the base of a pine tree or in its stump.

If desired, the resulting vinyl acetate can be polymerized to produce polyvinylacetate homopolymer, or it can be copolymerized with another monomer, such as a bio-based monomer, such as bio-ethylene. This polymer (or an EVA copolymer) can be hydrolyzed to a desired extent (to remove some or most of the dangling acetate groups in the polymer and replacing some or most of them by dangling hydroxyl groups), for example, 5, 10, 15, 25, 50, 75, 99 percent hydrolyzed or essentially completely hydrolyzed, to produce high modern carbon content vinyl alcohol polymers or polyvinyl alcohol-type polymers. The resulting polyvinyl alcohols or polyvinyl alcohol-type polymers can be functionalized by reacting the hydroxyl groups with various complementary groups, such as aldehydes and ketones. For example, polyvinyl butyral or polyvinyl formal can be produced. Many polyvinyl alcohol polymers and copolymers are degradable polymers and have many large volume uses, including in adhesives, emulsion polymerization where surface active materials may be desirable, in films and packaging, such as for use as an oxygen barrier layer in a multi-layer package, in cementing, in paper making and finishing, textiles, building and construction and in personal care products.

Thus, in certain embodiments, the present invention provides materials and methods for manufacturing polymers (e.g., polyethylene, including, for example, low density, high density, or linear low density polyethylene) from biomass. The biomass can be, e.g., sugar from sugarcane, starchy biomass, cellulosic biomass, or lignocellulosic biomass such as agricultural residues, woody biomass, municipal waste, oilseeds/cakes, and seaweed. For example, in some embodiments, the biomass material comprises a wood, a grass, or an agricultural residue. The biomass material may be corn cob. Accordingly, using certain biomass materials to produce bio-based ethylene and other products (e.g., bio-based polyethylene) makes use of biomass waste products while reducing the consumption of fossil fuels. In some embodiments, the bio-based ethanol is produced from a mixture of starchy and lignocellulosic biomass, such as grain-cob biomass or grain intermixed with corn stover. In certain instances, a high lignin content residue remaining after extracting sugars from the biomass can be burned to produce heat and power to reduce the global warming potential of the process by, among other things, utilizing a renewable and sustainable energy source.

Some embodiments of the present invention relate to compositions comprising bio-based ethylene and/or products produced or derived from bio-based ethylene (such as, e.g., vinyl chloride monomer, vinyl acetate monomer, 1,2-dichloroethane (commonly known as ethylene dichloride (EDC)), polyvinyl chloride, polyethylene, etc.), wherein the percent modern carbon (pMC) of the composition is at least about 50 pMC, at least about 60 pMC, at least about 70 pMC, at least about 80 pMC, at least about 90 pMC, or at least about 100 pMC. In certain embodiments, the pMC is at least about 90 pMC or at least about 100 pMC.

In certain embodiments, the present invention provides bio-based ethylene and products and compositions produced or derived therefrom, such as, e.g., vinyl chloride monomer, polyvinyl chloride, 1,2-dichloroethane (commonly known as ethylene dichloride (EDC)) and polyethylene. Blends of the bio-based ethylene, or products or compositions produced or derived therefrom, may be blended with one or more fossil-fuel derived products or compositions. For example, bio-based PVC and fossil-fuel derived PVC may be blended in equal proportions in order to, for example, balance cost and environmental concerns, such as GWP. In some embodiments, the bio-based ethylene and products and compositions produced or derived therefrom, or blends of such with fossil-fuel derived products or compositions can have, for example, a pMC of at least about 50 pMC, at least about 60 pMC, at least about 70 pMC, at least about 80 pMC, at least about 90 pMC, at least about 95 pMC or more, e.g., at least about 97, 98, 99 or more pMC, such as nearly or about 100 pMC. In preferred embodiments, such bio-based products or compositions or blends with fossil-fuel derived compositions, have a modern carbon content of greater than about 95 percent, such as greater than about 96, 97, 98, 98.5, 99 or greater, e.g., greater than 99.5 pMC.

Embodiments of the present invention also provide polymers, copolymers or polymer blends of bio-based ethylene (e.g., bio-based polyethylene, bio-based ethylene-vinyl acetate, bio-based ethylene-acrylic acid and bio-based ethylene-methacrylic acid), in which the percent modern carbon of the polymers, copolymers or polymer blends, e.g., intimate melt blends, is at least about 20 pMC, at least about 30 pMC, at least about 40 pMC, at least about 50 pMC, at least about 60 pMC, at least about 70 pMC, at least about 80 pMC, at least about 90 pMC, or more, such as at least about 95 pMC or more, such as at least about 96, 97, 98 pMC or more, such as at least about 99 pMC. In particular embodiments, the pMC is nearly or about 100 pMC. In copolymers, the percent modern carbon can be generally increased by having all or most of the co-monomers of a copolymer being bio-based. In blends, the percent modern carbon can be generally increased by having all or most of the polymers in the blend being bio-based.

In some embodiments, the present invention provides bio-based polyethylene and products derived therefrom, wherein the pMC of the bio-based polyethylene, or of the products derived therefrom, is/are at least about 50 pMC, at least about 60 pMC, at least about 70 pMC, at least about 80 pMC, at least about 90 pMC, at least about 95, 95, 97 or 98 pMC. In some instances, the bio-based polyethylene, or of the products derived therefrom, is/are nearly or about 100 pMC.

In certain embodiments, the present invention provides compositions comprising bio-based ethylene and/or products derived therefrom, such as, e.g., vinyl chloride monomer, vinyl acetate monomer, 1,2-dichloroethane (commonly known as ethylene dichloride (EDC)), polyvinyl chloride and polyethylene. In such embodiments, the percent bio-based carbon of the composition is at least about 50%, at least about 60%, at least about 70%, at least about 80%, at least about 90%, at least about 95%, or nearly or about 100%. In particular embodiments, the carbon in the composition is 100% bio-based carbon.

In certain embodiments, the present invention provides bio-based ethylene and products produced or derived therefrom (such as, e.g., vinyl chloride monomer, vinyl acetate monomer, 1,2-dichloroethane (commonly known as ethylene dichloride (EDC)), polyvinyl chloride, polyethylene, etc.), wherein the percent bio-based carbon in the bio-based ethylene, or in the products produced or derived therefrom, is at least about 50%, at least about 60%, at least about 70%, at least about 80%, at least about 90%, at least about 95%, at least about 98%, or about 100%. In particular embodiments, the carbon is 100% bio-based carbon.

In some embodiments, the present invention provides polymers or copolymers of bio-based ethylene (e.g., bio-based polyethylene, bio-based ethylene-vinyl acetate, bio-based ethylene-acrylic acid, bio-based ethylene-methacrylic acid, etc.), wherein the percent bio-based carbon of the polymers or copolymers is at least about 50%, at least about 60%, at least about 70%, at least about 80%, at least about 90%, at least about 95%, at least about 98%, or about 100%. In particular embodiments, the carbon in the polymer or copolymer is 100% bio-based carbon.

In further embodiments, the present invention provides bio-based polyethylene and products derived therefrom, wherein the percent bio-based carbon in the bio-based polyethylene or in the products derived therefrom is at least about 50%, at least about 60%, at least about 70%, at least about 80%, at least about 90%, at least about 95%, or about 100%. In particular embodiments, the carbon is 100% bio-based carbon.

Copolymers of the bio-based ethylene (or of blends of bio-based ethylene and fossil fuel-based ethylene) can be formed with one or more of the following monomers, which may be bio-based or fossil fuel-based: styrene, propylene, butylene, butadiene, isoprene, and an α-olefin, such as a C4-C22 α-olefin with an even number or an odd number of carbon atoms, such as one or more normal linear or branched alpha olefins, such as 1-hexene or 1-octene. The resulting copolymers may be considered polyolefin elastomers (POE) and can be very useful in consumer goods, for example, adding a “soft touch” to any product. In some embodiments, such polyolefin elastomers (POE), can be, for example, a styrene-ethylene-butylene-styrene copolymer (SEBS), for example, having random or regular soft and hard blocks. In any of these copolymers, in each instance, the ethylene can be entirely bio-based or a blend of bio-based and fossil fuel-based ethylene, and any co-monomer can be entirely bio-based, entirely fossil fuel-based or a blend of bio-based and fossil-fuel based. Any such copolymer or blends, such as those formed by intimate melt blending, can have, for example, a modern-carbon content of greater than about 10 percent, such as greater than about 15, 20, 25, 30, 40, 50 percent or more, e.g., greater than about 60, 70, 80, 90 percent or more, such as greater than about 95 percent modern carbon.

In any embodiment described herein that utilizes bio-based ethylene in its production, such as 1-octene, caprylyl glycol, 2-chloroethanol, the bio-based polymers or copolymers made, at least in part, with bio-based ethylene (e.g., bio-based polyethylene or SEBS) or blends of such polymers, as described herein, the bio-based ethylene utilized can be made, for example, from bio-based ethanol according to the methods described herein. The bio-based ethanol can be produced from a variety of modern carbon biomass sources, including but not limited to cellulosic or lignocellulosic biomasses, such as: paper, paper products, paper waste, paper pulp, pigmented papers, loaded papers, coated papers, filled papers, magazines, printed matter, printer paper, polycoated paper, card stock, cardboard, paperboard, cotton, wood, particle board, forestry wastes, sawdust, aspen wood, wood chips, grasses, switchgrass, miscanthus, cord grass, reed canary grass, grain residues, rice hulls, oat hulls, wheat chaff, barley hulls, agricultural waste, silage, canola straw, wheat straw, barley straw, oat straw, rice straw, jute, hemp, flax, bamboo, sisal, abaca, corn cobs, corn stover, soybean stover, corn fiber, alfalfa, hay, coconut hair, sugar processing residues, bagasse, beet pulp, agave bagasse, algae, seaweed, manure, sewage and agricultural or industrial waste that includes lignocellulose or cellulose. Various starch-rich materials, sugar-rich materials and sugars can also be utilized to produce the bio-ethanol, such as root vegetables, grains, and various fruits and vegetables. Examples include arracacha, buckwheat, banana, barley, cassava, kudzu, oca, sago, sorghum, potato, sweet potato, taro, yams, beans, favas, lentils, peas, sucrose, fructose and high fructose corn syrup, such as off-specification high fructose corn syrup. Mixtures of any of these sugars or derived sugars obtained from any of these modern carbon biomass sources can be utilized. In some embodiments, all of the sugars of the biomass source (such as xylose, glucose, and arabinose) are utilized to make the bio-based ethanol. Alternatively, one of the sugars (such as glucose) is converted to ethanol, leaving the other sugar or sugars available for use in other products, for example xylose for producing xylitol or xylose for producing succinic acid. Such high value coproducts allow for the production of less expensive bio-based ethanol, effectively subsidizing its production.

Accordingly, the present invention also provides methods for producing bio-based ethylene and other products that can be made from such bio-based ethylene (e.g., bio-based polyethylene, 1-octene and other oligomers of ethylene, caprylyl glycol, 2-chloroethanol or ethylene oxide). In certain embodiments, the present invention provides methods for producing bio-based ethylene comprising: (a) obtaining bio-based ethanol from a biomass material; and (b) catalytically dehydrating the bio-based ethanol to generate bio-based ethylene. This generated bio-based ethylene can be further processed to produce other products—e.g., the bio-based ethylene can be polymerized to produce bio-based polyethylene. Such bio-based polyethylene may be low density, such as linear low density bio-based polyethylene, medium density, or high density bio-based polyethylene. Such products can be produced in a sustainable manner at the lowest cost and lowest carbon footprint.

In certain embodiments, the bio-based ethanol is obtained from a biomass material that is non-food biomass (e.g., agricultural or municipal waste). In some embodiments, the biomass material from which the bio-based ethanol is produced comprises lignocellulosic material. In particular embodiments, the biomass material comprises corn cob, such as corn cobs from seed corn.

In certain embodiments of the methods described herein, the catalyst used in the dehydration reaction (Catalyst One or CAT 1, as used herein) comprises a metal oxide, a silico-aluminate, a silico-aluminophosphate, or a heteropoly acid.

For example, in some embodiments, Catalyst One comprises Al₂O₃, TiO₂—Al₂O₃, SiO₂, SiO₂—Al₂O₃, ZrO₂, WO₃, ZnO/Al₂O₃, MgO—Al₂O₃/SiO₂, USY (ultrastable Y zeolite), or ZSM5. In embodiments where the catalyst comprises ZSM5, the ZSM5 may have a Si/Al ratio that is 20:1 to 360:1 (such ratios are typically denoted as “20” or “360”); in other embodiments, the ZSM5 has a Si/Al ratio of 19:1 (or 19). For example, the Si/Al ratio can be greater the about 18, 19, 20, or more, such as greater than about 20, 22, 28, 32, 36, 40, or more, such as greater than about 50, 60, 70, 80, 90, 100, 150, or more, such as greater than about 200, 250, 300 or more, such as greater than about 350. In other embodiments, the Si/Al ratio is between about 18 and 360, such as between about 20 and 300, between about 22 and 250, between about 24 and 200, between about 30 and 170 or between about 35 and 150.

In certain embodiments, any Catalyst One described herein can be modified with a transition metal, such as lanthanum. In some embodiments, the catalyst is modified to contain between about 0.05 and about 15 percent by weight of one or more lanthanide elements, such as between about 0.05 and about 10 percent by weight, between about 0.1 and about 7.5 percent by weight or between about 0.5 and about 5 percent by weight of a lanthanide element (elements 57 through 71, inclusive). In particular embodiments, the lanthanide element is lanthanum, and the catalyst includes between about 0.5 and about 5 percent by weight lanthanum, such as between about 0.7 and about 2.5 percent by weight, or between about 0.8 and about 1.5 percent by weight lanthanum. In a specific embodiment, the catalyst contains about 1% by weight lanthanum.

In further embodiments, the Catalyst One is a zeolite treated with H₃PO₄. Catalyst One may be prepared in certain embodiments by slowly adding a calculated amount of H₃PO₄ aqueous solution to zeolite with constant stirring. The resultant solid may be kept in a sealed beaker for 3 hours. The impregnated solid catalyst may be dried at 120° C. overnight and then calcined at 400° C. for 5 hours.

Methods of the present invention also include embodiments where Catalyst One comprises molybdophosphoric acid or tungstophosphoric acid.

In certain embodiments, the catalytic dehydration reaction using Catalyst One converts at least about 50%, at least about 60%, at least about 70%, at least about 80%, at least about 90%, at least about 95%, at least about 98%, at least about 99%, or nearly 100% of the bio-based ethanol.

In certain embodiments, the catalytic dehydration reaction utilizing Catalyst One generates bio-based ethylene at a mole percent that is at least about 50 mole percent, at least about 60 mole percent, at least about 70 mole percent, at least about 80 mole percent, at least about 90 mole percent, at least about 95 mole percent, at least about 98 mole percent, or about 100 mole percent.

In certain embodiments, the catalytic dehydration reaction utilizing Catalyst One is conducted at a temperature that is from about 250° C. to about 500° C. In further embodiments, the temperature is within the range of about 325-425° C.; for example, the temperature may be about 400° C. In other embodiments, generating the bio-based ethylene is conducted at a temperature that is below about 300° C.

In certain embodiments, the catalytic dehydration reaction utilizing Catalyst One is conducted in a tubular reactor, wherein the bio-based ethanol is added to the reactor in liquid form. In such embodiments, the flow rate of the bio-based ethanol is about 0.2 ml/min to about 0.5 ml/min. In some embodiments, the flow rate of bio-based ethanol is about 0.25 ml/min; in other embodiments, the flow rate is about 0.3 ml/min. The flow rate of bio-based ethanol may also be expressed in terms of liquid hourly space velocity (LHSV), which is a ratio of liquid volume per hour to the volume of catalyst. In some embodiments, the LHSV of the bio-based ethanol may be between about 1 h⁻¹ to about 2.5 h⁻¹. In some embodiments, the LHSV of the bio-based ethanol may be about 1.25 h⁻¹; in other embodiments, the LHSV of the bio-based ethanol may be about 1.5 h⁻¹.

In any of the embodiments herein, the biomass material used to produce the bio-based ethanol may comprises any one or more of the following cellulosic or lignocellulosic biomass materials: paper, cardboard, cotton, wood, particle board, forestry wastes, sawdust, aspen wood, wood chips, grasses, switchgrass, miscanthus, cord grass, reed canary grass, grain residues, rice hulls, oat hulls, wheat chaff, barley hulls, agricultural waste, silage, canola straw, wheat straw, barley straw, oat straw, rice straw, jute, hemp, flax, bamboo, sisal, abaca, corn cobs, corn stover, soybean stover, corn fiber, alfalfa, hay, coconut hair, sugar processing residues, bagasse, beet pulp, agave bagasse, algae, seaweed, manure, sewage, offal, agricultural or industrial waste, arracacha, buckwheat, banana, barley, cassava, kudzu, oca, sago, sorghum, potato, sweet potato, taro, yams, beans, favas, lentils, or peas.

In certain embodiments, the bio-based ethanol is obtained from a biomass material that is or that comprises a wood, a grass, or an agricultural residue, or a combination of one or more these materials. In some embodiments, the biomass material is or comprises corn cob.

The methods of the present invention can produce bio-based ethylene and bio-based polyethylene, and other bio-based products that can be made with bio-based ethylene, wherein the percent modern carbon (pMC) value of such bio-based ethylene, bio-based polyethylene, or other bio-based product is at least about 50 pMC, at least about 60 pMC, at least about 70 pMC, at least about 80 pMC, at least about 90 pMC, at least about 95 pMC, at least about 98 pMC, or at least about 100 pMC.

Similarly, the methods of the present invention can produce bio-based ethylene and bio-based polyethylene, and other bio-based products that can be made with bio-based ethylene, wherein the percent bio-based carbon value of such bio-based ethylene, bio-based polyethylene, or other bio-based product is at least about 50%, at least about 60%, at least about 70%, at least about 80%, at least about 90%, at least about 95%, at least about 98%, or about 100%.

In certain embodiments, the invention is directed to a composition comprising a mixture of bio-based α-olefins, wherein said bio-based α-olefins contain four, six, eight, ten, twelve, or more carbons per α-olefin, and wherein the percent modern carbon (pMC) of the composition is at least about 50 pMC, at least about 60 pMC, at least about 70 pMC, at least about 80 pMC, at least about 90 pMC, at least about 95 pMC, at least about 98 pMC, or at least about 100 pMC. In certain embodiments, said bio-based α-olefins comprise 1-butene, 1-hexene, 1-octene, 1-decene, or 1-dodecene.

In certain embodiments, the invention is directed to a composition comprising a bio-based α-olefin, wherein said bio-based α-olefin contains four carbons per α-olefin, and wherein the percent modern carbon (pMC) of the composition is at least about 50 pMC, at least about 60 pMC, at least about 70 pMC, at least about 80 pMC, at least about 90 pMC, at least about 95 pMC, at least about 98 pMC, or at least about 100 pMC. In certain embodiments, said bio-based α-olefin comprises 1-butene, 1-hexene, 1-octene, 1-decene, or 1-dodecene.

In certain embodiments, the invention is directed to methods for making and compositions comprising one or more bio-based 1,2-alkyldiols, e.g., those made by dihydroxylation of olefins, such as alpha olefins produced from oligomerization of bio-based ethylene or mixtures of bio-based ethylene and fossil fuel-based ethylene. In such embodiments, the one or more 1,2-alkyldiol compositions can contain, for example, an even number of carbon atoms, such as four, six, eight, ten, twelve, fourteen, sixteen, eighteen or more carbons atoms per 1,2-alkyldiol. Generally, it is desirable to maximize the modern carbon or naturally derived content of such compositions by utilizing sustainable materials as carbon sources on balance with the cost of such compositions. In some implementations, such compositions have a percent modern carbon (pMC) content that is at least about 50 pMC, or more, such as at least about 60 pMC, 70 pMC, 80 pMC or more, such as at least about 85 pMC, 87.5 pMC, 90 pMC, 92.5 pMC, 96 pMC or more, such as at least about 98 pMC. In specific embodiments, the compositions are nearly or about 100 pMC. In certain embodiments, the bio-based 1,2-alkyldiol comprises one or more of 1,2-butanediol, 1,2-hexanediol, 1,2-octanediol, 1,2-decanediol, or 1,2-dodecanediol, each being in any stereoisomeric form, R-, S- or R/S-. In a specific embodiment, the composition includes a blend of 1,2-octanediol (caprylyl glycol) and 1,2-hexanediol.

In certain embodiments, the invention is directed to a cream, jelly, ointment, paste, cerate, chrism, cosmetic, demulcent, emulsion, essence, liniment, salve, unction, unguent, or moisturizer comprising one or more bio-based 1,2-alkyldiols or blends of bio-based 1,2-alkyldiols and fossil fuel-based 1,2-alkyldiols. Such one or more 1,2-alkyldiols can, for example, contain four, six, eight, ten, twelve or more carbons per 1,2-alkyldiol, and can have a high natural-origin, modern carbon content, for example, having a percent modern carbon (pMC) of at least about 50 pMC, such as at least about 60, 70, 80, 90 pMC or more, e.g., at least about 91, 92, 93, 94, 95, 96, 97, 98, 99 or more pMC. In preferred embodiments, the 1,2-alkyldiols are nearly or about 100 pMC. In certain embodiments, the 1,2-alkyldiols include one or more of the following 1,2-butanediol, 1,2-hexanediol, 1,2-octanediol, 1,2-decanediol, or 1,2-dodecanediol.

In certain embodiments, the invention is directed to a cream, jelly, ointment, paste, cerate, chrism, cosmetic, demulcent, emulsion, essence, liniment, salve, unction, unguent, or moisturizer that includes blends of one or more bio-based preservatives, or blends of one or more bio-based preservatives and fossil fuel-derived preservatives. Such preservatives can be, for example, antimicrobial, for example, kill or inhibit growth of bacteria, fungal or yeasts, or antiviral. For example, one preservative system includes one or more bio-based 1,2-alkyldiols, such as 1,2-hexanediol or 1,2-octanediol (caprylyl glycol), in combination with a phenolic preservative, such as phenoxyethanol. If desired, the phenoxyethanol may be 100 percent natural-origin, as described herein, or it may be fossil fuel derived. For example, the preservative system may be about 100 pMC and may include a caprylyl glycol/phenoxyethanol ratio of between about 1:1 to about 10:1, such as between about 2:1 to about 9:1 or between about 3:1 to about 8:1. As described herein, phenoxyethanol can be produced, for example, by first producing phenol by de-methoxylation of guaiacol and then reacting the phenol (or phenoxide) with 2-chloroethanol or ethylene oxide produced from bio-based ethylene, as described herein. Combination preservative systems can act synergistically for a more powerful action. The combination naturally derived, bio-based preservative can have a high modern carbon or natural-origin content, for example, a percent modern carbon (pMC) of at least about 20 pMC, at least about 30 pMC, at least about 40 pMC, at least about 50 pMC, at least about 60 pMC, at least about 70 pMC, at least about 80 pMC, at least about 90 pMC, or even about 100 pMC.

In certain embodiments, the invention is directed to a composition comprising a bio-based vinyl acetate monomer, wherein the percent modern carbon (pMC) of the composition is at least about 20 pMC, at least about 30 pMC, at least about 40 pMC, at least about 50 pMC, at least about 60 pMC, at least about 70 pMC, at least about 80 pMC, at least about 90 pMC. In specific embodiments, the vinyl acetate monomer is nearly or about 100 pMC. A high modern carbon content vinyl acetate monomer may be produced by reaction of bio-based ethylene with bio-based acetic acid, for example, from oxidized bio-based ethanol, with oxygen in the presence of a catalyst, such as a palladium catalyst, as described herein.

In certain embodiments, the invention is directed to a composition comprising a bio-based ethylene-vinyl acetate (EVA) copolymer, wherein the percent modern carbon (pMC) of the composition is at least about 20 pMC, at least about 30 pMC, at least about 40 pMC, at least about 50 pMC, at least about 60 pMC, at least about 70 pMC, at least about 80 pMC, at least about 90 pMC, or even about 100 pMC. In particular embodiments, the pMC is at least about 50 pMC and in other particular embodiments the pMC is nearly or about 100 pMC. In certain embodiments, the mole percentage of ethylene monomer in the composition is in the range of about 70 percent to about 98 percent, such as between about 75 and about 95 percent, between about 78 and about 94 percent or between about 80 and about 92 percent. In certain embodiments, the mole percentage of vinyl acetate monomer in the composition is in the range of about 2 percent to about 30 percent, such as between about 3 and about 27 percent, between about 4 and about 25 percent or between about 7 and about 20 percent.

In certain embodiments, the invention is directed to a composition comprising a bio-based ethylene-vinyl acetate (EVA) foam. The bio-based EVA foam may be formed by foaming the bio-based EVA copolymer composition described above using conventional methods and blowing and/or foaming agents, such as, for example and without limitation, carbon dioxide, butane, or azodicarbonamide.

If desired, for example, to make a more wear resistant EVA, the bio-based EVA may be crosslinked, for example, by using an azo initiator or radiation, such as UV, gamma or an electron beam. When radiation is utilized to crosslink the EVA, the dose utilized can be, for example, between about 0.25 Mrad and about 20 Mrad, such as between about 0.5 Mrad and about 15 Mrad, or between about 1 Mrad and about 12 Mrad.

In certain embodiments, the invention is directed to a composition that may be utilized in the manufacture of footwear, such as, for example and without limitation, in the soles, midsoles, uppers, and/or bodies of sandals, boots, galoshes, loafers, slippers, moccasins, or athletic, running, leisure, walking, tennis, derby, oxford, slip-on, dress, or casual shoes. The composition may comprise one or more bio-based ethylene-vinyl acetate (EVA) copolymers or bio-based EVA foams wherein the percent modern carbon (pMC) of the composition is at least about 20 pMC, at least about 30 pMC, at least about 40 pMC, at least about 50 pMC, at least about 60 pMC, at least about 70 pMC, at least about 80 pMC, at least about 90 pMC, or about 100 pMC. In certain embodiments, the mole percentage of ethylene monomer in the bio-based EVA copolymer or bio-based EVA foam composition is between about 75 and about 95 percent, between about 78 and about 94 percent or between about 80 and about 92 percent. In certain embodiments, the mole percentage of vinyl acetate monomer in the composition is in the range of about 2 percent to about 30 percent, such as between about 3 and about 27 percent, between about 4 and about 25 percent or between about 7 and about 20 percent. In some embodiments, the density of the EVA foam is between about 0.2 and about 0.8 g/cm³, such as between about 0.3 and about 0.7 g/cm³ or between about 0.35 and about 0.6 g/cm³.

In certain embodiments, the invention is directed to a method of making a bio-α-olefin comprising the step of oligomerizing bio-based ethylene, or blends of bio-based ethylene and fossil fuel-based ethylene, to produce a desired modern carbon content, in the presence of a catalyst (“Catalyst Two” or “CAT 2,” described further herein) to produce one or more bio-α-olefins. In certain embodiments, said α-olefin contains four, six, eight, ten, twelve or more carbon atoms. In certain embodiments, said α-olefin comprises 1-octene. In certain embodiments, said Catalyst Two is in a +3 oxidation state, such as Cr in +3 oxidation state, Mo in a +3 oxidation state or W in a +3 oxidation state, for example, chromium(III) acetylacetonate, Cr(acac)₃, chromium(III) nitrate, chromium(III) acetate, chromium(III) oxide and chromium(III) chloride. In embodiments, the resulting one or more bio-α-olefins have a percent modern carbon (pMC) content that is at least about 20 pMC, at least about 30 pMC, at least about 40 pMC, at least about 50 pMC, at least about 60 pMC, at least about 70 pMC, at least about 80 pMC, at least about 90 pMC, or even about 100 pMC.

In certain embodiments, the invention is directed to a method of making a bio-1,2-diol, wherein the method comprises oxidizing and hydrolyzing one or more bio-α-olefins in the presence of an oxidizing agent to yield one or more bio-1,2-diols. In certain embodiments, said oxidizing agent includes aqueous KMnO₄, OsO₄, and H₂O₂. In certain embodiments, said bio-1,2-diol contains four, six, eight, ten, twelve or more carbons. In certain embodiments, said bio-1,2-diol comprises bio-1,2-octanediol. Other methods of dihydroxylation include the Milas method, Upjohn method, Sharpless method, Prevost-Woodward method and the Sudali modified Prevost-Woodward method. Osmium tetroxide can be difficult to work with due to its toxicity, so it can be advantageous to make the dihydroxylation catalyst in-situ, for example, by using sodium periodate and ruthenium trichloride, producing ruthenium tetroxide in-situ. In embodiments, the one or more bio-1,2-diols have a percent modern carbon (pMC) content that is at least about 20 pMC, at least about 30 pMC, at least about 40 pMC, at least about 50 pMC, at least about 60 pMC, at least about 70 pMC, at least about 80 pMC, at least about 90 pMC, or even about 100 pMC.

In certain embodiments, the invention is directed to a method of making a bio-based phenoxyethanol. In such embodiments, the method comprises oxidizing bio-based ethylene, or a mixture of bio-based ethylene and fossil fuel-based ethylene, in the presence of oxygen and a catalyst (e.g., a silver-based catalyst) to yield bio-based ethylene oxide, such as a fully bio-based ethylene oxide. The bio-based ethylene oxide is further reacted with phenol in the presence of, for example, an alkali-metal hydroxide, to yield bio-based phenoxyethanol. In other embodiments, the invention is directed to a method of making a bio-based phenoxyethanol. The method comprises reacting bio-based ethylene, or a blend of bio-based ethylene and fossil fuel-based ethylene, with a hypohalous acid (e.g., hypochlorous acid) to yield a bio-based halohydrin (e.g., 2-chloroethanol). The bio-based halohydrin is further reacted with a phenoxide ion source to yield bio-based phenoxyethanol. The halohydrin may also be produced by reaction of the bio-based ethylene with trichloroisocyanuric acid in aqueous acetone solutions. Any of these halohydrin methods can be applied to any bio-based alpha olefin described herein, producing the corresponding valuable bio-based halohydrins. In embodiments, the phenoxyethanol, ethylene oxide, 2-chloroethanol, any one or more alpha olefins and any one or more halohydrins can have a percent modern carbon (pMC) content that is at least about 20 pMC, at least about 30 pMC, at least about 40 pMC, at least about 50 pMC, at least about 60 pMC, at least about 70 pMC, at least about 80 pMC, at least about 90 pMC, or even about 100 pMC.

In certain embodiments, the invention is directed to a method of making a bio-based vinyl acetate monomer. The method includes reacting bio-based ethylene, or blends of bio-based ethylene and fossil fuel-based ethylene, with acetic acid, for example, a bio-based acetic acid and oxygen in the presence of a catalyst (“Catalyst 3” or “CAT 3,” described further herein). In certain embodiments, the acetic acid may be a bio-based acetic acid, such as, for example, bio-acetic acid obtained from the hydrocarboxylation of bio-based ethylene or bio-acetic acid from over oxidation of bio-ethanol. However, this is not required, and the acetic acid may be obtained from any source. In certain embodiments, the catalyst may be a palladium-based catalyst, such as Pd—Au catalyst with a potassium acetate activator impregnated on silica particles. In other embodiments, the catalyst may be palladium chloride, palladium acetate, or copper chloride. In embodiments, the bio-based vinyl acetate monomer can have a percent modern carbon (pMC) content that is at least about 20 pMC, at least about 30 pMC, at least about 40 pMC, at least about 50 pMC, at least about 60 pMC, at least about 70 pMC, at least about 80 pMC, at least about 90 pMC, or even about 100 pMC.

In certain embodiments, the invention is directed to a method of making a bio-based ethylene-vinyl acetate (EVA) copolymer. The method includes reacting bio-based ethylene, or a mixture of bio-based ethylene and fossil fuel-based ethylene and vinyl acetate, for example, a fully bio-based vinyl acetate in the presence of a composite oxidation-reduction (“redox”) catalyst (“Catalyst 4” or “CAT 4,” described further herein). The composite redox catalyst may include a peroxygen compound and one or more of a metal salt, a heavy metal ion which may exist in more than one valence state, and an organic reducing agent. In other embodiments, the composite redox catalyst may comprise triethylaluminum, zinc chloride, and carbon tetrachloride (AlEt₃-ZnCl₂—CCl₄). In some embodiments, the bio-based ethylene-vinyl acetate (EVA) copolymer can have a percent modern carbon (pMC) content that is at least about 20 pMC, at least about 30 pMC, at least about 40 pMC, at least about 50 pMC, at least about 60 pMC, at least about 70 pMC, at least about 80 pMC, at least about 90 pMC, or even about 100 pMC.

### DESCRIPTION OF THE DRAWINGS

FIG. 1 depicts a reaction scheme for the production of a bio-1,2-alkyldiol from treated biomass. In particular, the biomass, such as corn cob, is first subjected to ionizing radiation in the form of an electron beam, or acid or steam explosion to reduce the recalcitrance of the biomass. The treated biomass is then treated enzymatically to generate high modern carbon content, natural-origin bio-sugars, such as bio-xylose and bio-glucose, from the biomass. These sugars could come from, for example, corn cob, corn stover, or in the alternative, these sugars can come from any starchy materials, such as from corn kernels or cassava, a sucrose source, such as from sugarcane, seaweed or any biomass that includes low molecular weight sugars (e.g., sugarcane) or is a source of low molecular sugars after suitable pretreatment, such as saccharification of starchy materials, for example, using amylases, or ebeam treatment of lignocellulosic materials followed by treatment with enzyes, for example, cellulases and hemicellulases, such as those produced during submerged fermentation by various strains of Trichoderma reesei, such as those presented by Xyleco in U.S. EPA MCAN J-19-0001. Any combination of biomass can be utilized, for example combinations of starchy materials and lignocellulosic materials. The bio-sugars are then fermented in the presence of yeast to generate natural-origin, bio-based ethanol. In particular embodiments, the yeast utilized, e.g., is a GM organism, that can metabolize both C5 and C6 sugars to produce ethanol. In other embodiments, only the glucose is converted to ethanol, leaving xylose available as an additional product. The bio-based ethanol is then converted to bio-based ethylene in the presence of a catalyst (CAT 1), for example, a dehydration catalyst, for example, one or more zeolites as further described herein. The bio-based ethylene is then subjected to oligomerization in the presence of a catalyst (CAT 2), for example, a heterogenous or a homogeneous catalyst, for example, a chromium catalyst, such as a chromium catalyst in the 3+ oxidation state, with phosphine ligands, for example, a bi- or tri-dentate phosphine ligand, to generate various natural-origin, high modern carbon content bio-α-olefins that may contain four, six, eight, ten, twelve, or more carbon atoms, e.g., 14, 16, 18 or 20 carbon atoms. In the case where the bio-α-olefin is bio-1-octene, it may be isolated and converted to bio-1,2-octanediol (caprylyl glycol) by oxidation and hydrolysis, which can occur in the same reaction vessel or in two separate reaction vessels. Non-limiting examples of oxidants that may be used are, sodium periodate and ruthenium trichloride, producing ruthenium tetroxide in-situ, and KMnO₄, OsO₄, and H₂O₂, for example. In FIG. 1, the chiral center in bio-1,2-octanediol (caprylyl glycol) is indicated by an asterisk (*).

FIG. 2 is a schematic representation of some of the many chemical compounds that can be obtained, and reactions that can be undertaken, starting from the bio-α-olefins or bio-based ethylene of the invention, for example, hydrohalogenation (Markovnikov as shown or anti-Markovnikov), hydrogenation, epoxidation, alkylation, isomerization, carboalkoxylation, dihydroxylation, ozonolysis, hydroformylation, hydrocarboxylation, hydroamination, polymerization or copolymerization, for example, to produce linear low density polyethylene and olefin metathesis, which allows for the production of compounds of odd numbers of carbon atoms from even carbon number alpha olefins. In certain embodiments, R1 and R2 may define families of compounds beyond alpha olefins and may refer to a non-specific sidechain (any atom that is not hydrogen) or any carbon-containing chain of any sort (e.g., alkyl, alkenyl or aryl). In these embodiments, R1 and R2 may be any carbon-containing chain, ring, or molecule portion. In other embodiments, R1 may be a hydrogen atom (depicting ethylene). In these embodiments, FIG. 2 schematically depicts some of the many chemical compounds that can be obtained of natural origin and of a desired pMC, and reactions that can be undertaken, starting from the bio-based ethylene of the invention. FIG. 2 does not exhaustively depict all of the chemical compounds that can be obtained or reactions that can be undertaken starting from the bio-α-olefins or bio-based ethylene of the invention, and those skilled in the art will appreciate that many additional reactions are possible. Those of skill in the art will also appreciate from FIG. 2 that mixtures of bio-based ethylene or bio-alpha olefins with their analogous fossil fuel-based compounds allow for making compositions having any desired pMC content, from low levels, such as 1 to 2 pMC to medium levels, such as 40 to 60 pMC, to higher levels, such as 80 to 99 pMC, or nearly 100 pMC.

FIG. 3 is a further schematic representation of some of the many chemical compounds that can be obtained, and reactions that can be undertaken, starting from the bio-α-olefins or bio-based ethylene of the invention, for example, Heck coupling, halogenation, hydroalkenylation, oxymercuration, Büchi acetone reaction, cyclopropanation, hydrophosphination, Diels-Alder reaction, hydroboration, hydroacylation, hydration, and halohydrin reactions, for example with HOX, where X is Cl or Br, or with an equivalent, such as with trichloroisocyanuric acid (swimming pool bleach), or tribromoisocyanuric acid. In certain embodiments, R1 and R2 may define families of compounds beyond alpha olefins and may refer to a non-specific sidechain (any atom that is not hydrogen) or any carbon-containing chain of any sort (e.g., alkyl, alkenyl or aryl). In these embodiments, R1 and R2 may include any carbon-containing chain, ring, or molecule. In other embodiments, R1 may be a hydrogen atom. In these embodiments, FIG. 3 schematically depicts some of the many chemical compounds that can be obtained, and reactions that can be undertaken, starting from the bio-based ethylene of the invention. FIG. 3 does not exhaustively depict all of the bio-based, high pMC chemical compounds that can be obtained or reactions that can be undertaken starting from the bio-α-olefins or bio-based ethylene of the invention, and those skilled in the art will appreciate that many additional reactions are possible. Those of skill in the art will also appreciate from FIG. 3 that mixtures of bio-based ethylene or bio-alpha olefins with their analogous fossil fuel-based compounds allow for making compositions having any desired pMC content, from low levels, such as 1 to 2 pMC to medium levels, such as 40 to 60 pMC, to higher levels, such as 80 to 99 pMC, or nearly 100 pMC.

FIG. 4 is a further schematic representation of a series of chemical reactions that can be undertaken starting from the bio-based ethanol of the invention so as to form bio-based vinyl acetate monomers (VA) and/or bio-based ethylene (E)-vinyl acetate (VA) copolymers (EVA). The bio-based ethanol is first converted to bio-based ethylene in the presence of a catalyst (CAT 1), for example, a dehydration catalyst such as one or more zeolites as further described herein. Bio-based vinyl acetate monomers are synthesized by reacting the resultant bio-based ethylene with acetic acid and oxygen in the presence of a catalyst (CAT 3), for example, a palladium-based catalyst such as a Pd—Au catalyst impregnated on spherical silica particles with potassium acetate. Other acceptable catalysts may include, without limitation, palladium chloride, palladium acetate, or copper chloride. Bio-based ethylene-vinyl acetate (EVA) copolymers may thereafter be made by reacting the bio-based vinyl acetate monomers with additional bio-based ethylene in the presence of a catalyst (CAT 4), for example, a composite oxidation-reduction (“redox”) catalyst comprising, for example, a peroxygen compound and one or more of a metal salt, heavy metal ion which may exist in more than one valence state, and an organic reducing agent. Alternatively, CAT 4 may comprise triethylaluminum, zinc chloride, and carbon tetrachloride (AlEt₃-ZnCl₂—CCl₄). Those of skill in the art will also appreciate from FIG. 4 that EVA copolymers having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

FIG. 5 is a schematic representation of a reaction pathway that may be undertaken in order to produce polyvinyl alcohol polymers with a high percent modern carbon (pMC). In certain embodiments, bio-based vinyl acetate homopolymers are hydrolyzed to a desired extent in order to produce high-pMC polyvinyl alcohol compounds.

FIG. 6 is a schematic representation of a reaction pathway that may be undertaken in order to produce ethylene vinyl alcohol compounds with a high percent modern carbon (pMC). In certain embodiments, bio-based ethylene-vinyl acetate copolymers are hydrolyzed to a desired extent in order to produce high-pMC ethylene vinyl alcohol compounds.

FIG. 7 is a further schematic representation of possible chemical reactions that polyvinyl alcohol compounds with a high percent modern carbon (pMC) may undergo. For example, in certain embodiments high-pMC polyvinyl alcohol compounds may be reacted with butyraldehyde to produce polyvinyl butyral. In other embodiments, high-pMC polyvinyl alcohol may be reacted with formaldehyde to produce polyvinyl formal.

FIG. 8 graphically depicts the results of a gas chromatography analysis of a sample obtained from the experiment described in Example 10 herein. A peak corresponding to 1-octene peak occurs at approximately 5 minutes of retention time, and reflects that a minimal amount of 1-octene was present in the analyzed sample.

FIG. 9 graphically depicts the results of a gas chromatography analysis and a mass spectral analysis of a sample obtained from the experiment described in Example 11 herein. Both the GC-FID and mass spectroscopy plots indicate large peaks corresponding to 1-octene, indicating a significant concentration of 1-octene in the analyzed samples.

FIG. 10 graphically depicts the results of a gas chromatography analysis and a mass spectral analysis of a sample obtained from the experiment described in Example 12 herein. Both the GC-FID and mass spectroscopy plots indicate large peaks corresponding to 1-octene, indicating a significant concentration of 1-octene in the analyzed samples.

FIG. 11 graphically depicts the results of a gas chromatography analysis and a mass spectral analysis of a sample obtained from the experiment described in Example 13 herein. Both the GC-FID and mass spectroscopy plots indicate large peaks corresponding to 1-octene, indicating a significant concentration of 1-octene in the analyzed samples.

FIG. 12 graphically depicts the results of a gas chromatography analysis and a mass spectral analysis of a sample obtained from the experiment described in Example 14 herein. Both the GC-FID and mass spectroscopy plots indicate large peaks corresponding to 1-octene, indicating a significant concentration of 1-octene in the analyzed samples.

FIG. 13 graphically depicts a ¹³C NMR spectrum of an EVA copolymer obtained from the experiment described in Example 16 herein. The solvent used was a 2:1 mixture of TCE and benzene-d6.

FIG. 14 is a schematic representation of an exemplary mass production process for the production of 1,2-octanediol (caprylyl glycol) from 1-octene. The raw materials in this process may be bio-based and have a high modern carbon content and pMC.

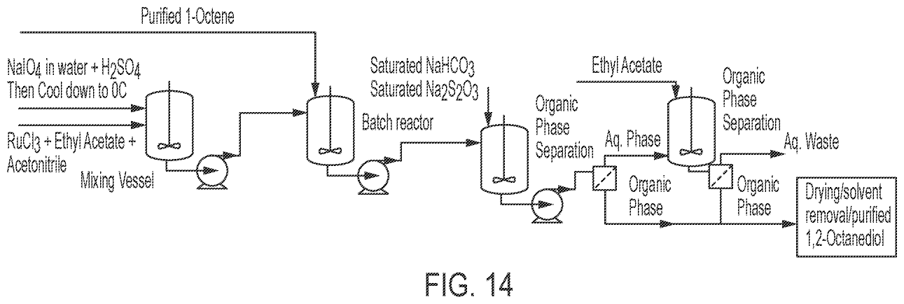

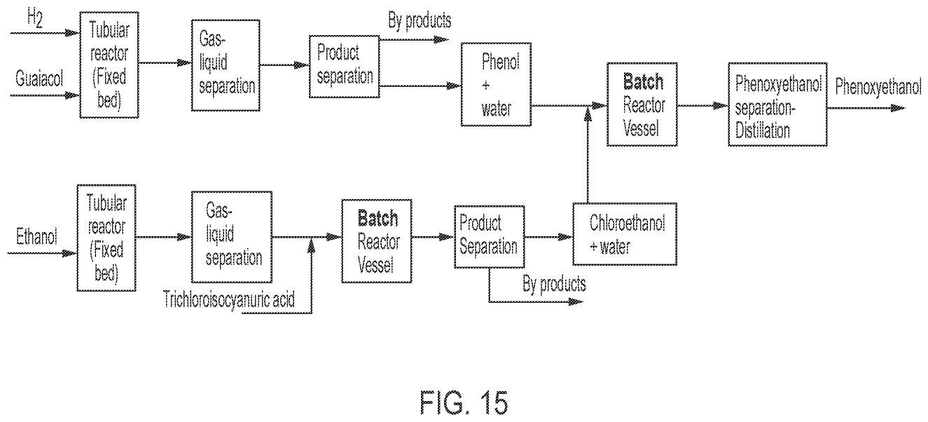

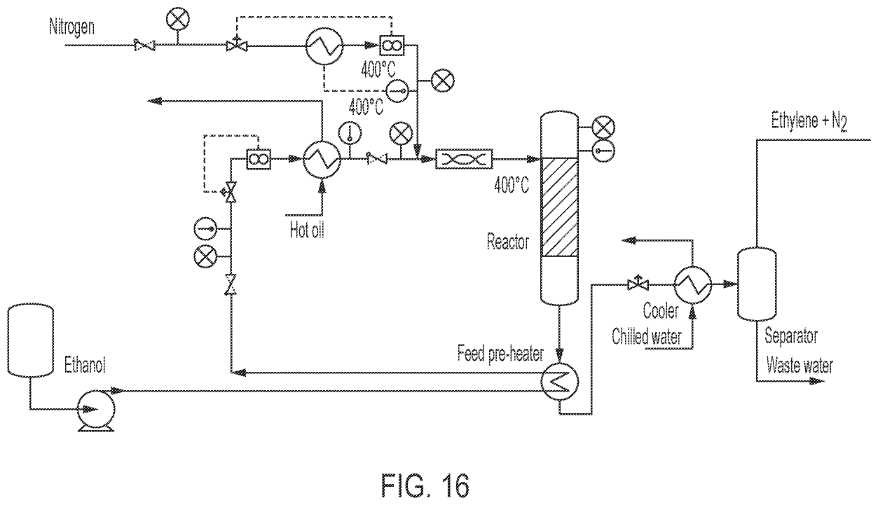

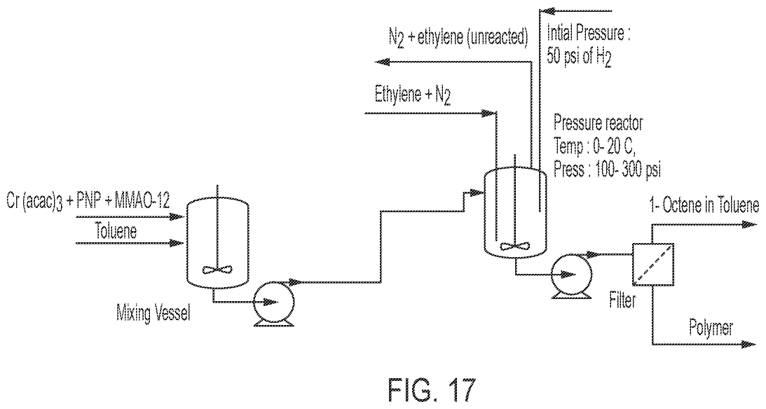

Therefore, the 1,2-octanediol (caprylyl glycol) that is produced may also be bio-based and have a high modern carbon content and pMC. Those of skill in the art will appreciate that blends of bio-based and fossil fuel-derived 1-octene may be utilized in order to balance costs, sustainability, and environmental concerns, including GWP. Those of skill in the art will also appreciate from FIG. 14 that bio-based 1,2-octanediol (bio-based caprylyl glycol) having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

FIG. 15 is a schematic representation of an exemplary mass production process for the production of phenoxyethanol from guaiacol and ethanol. The raw materials in this process may be bio-based and have a high modern carbon content and pMC. Therefore, the phenoxyethanol that is produced may also be bio-based and have a high modern carbon content and pMC. Those of skill in the art will appreciate that blends of bio-based and fossil fuel-derived guaiacol and ethanol may be utilized in order to balance costs, sustainability, and environmental concerns, including GWP. Those of skill in the art will also appreciate from FIG. 15 that bio-based phenoxyethanol having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

FIG. 16 is a schematic representation of an exemplary mass production process for the production of ethylene from ethanol. The raw materials in this process may be bio-based and have a high modern carbon content and pMC. Therefore, the ethylene that is produced may also be bio-based and have a high modern carbon content and pMC. Those of skill in the art will appreciate that blends of bio-based and fossil fuel-derived ethanol may be utilized in order to balance costs, sustainability, and environmental concerns, including GWP. Those of skill in the art will also appreciate from FIG. 16 that bio-based ethylene having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

FIG. 17 is a schematic representation of an exemplary mass-scale process of ethylene oligomerization to alpha-olefins. The raw materials in this process may be bio-based and have a high modern carbon content and pMC. Therefore, the 1-octene and polymer that is produced may also be bio-based and have a high modern carbon content and pMC. Those of skill in the art will appreciate that blends of bio-based and fossil fuel-derived ethylene may be utilized in order to balance costs, sustainability, and environmental concerns, including GWP. Those of skill in the art will also appreciate from FIG. 17 that bio-based 1-octene and polymer having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

### DETAILED DESCRIPTION OF THE INVENTION

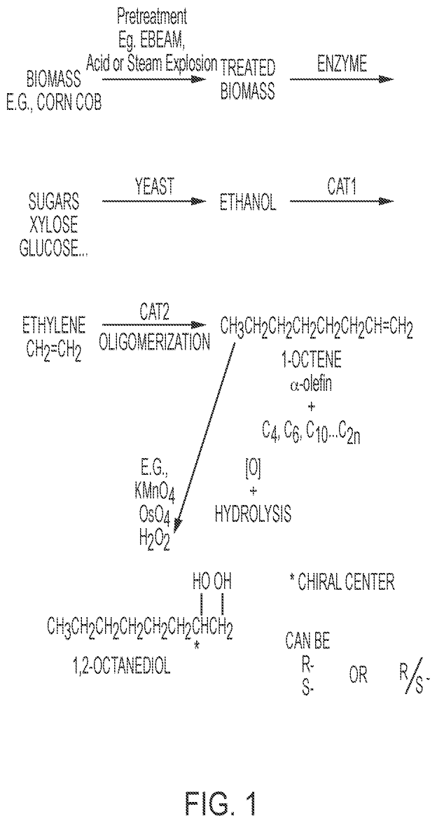

The present invention provides materials and methods for sustainably manufacturing a variety of bio-based ingredients that natural, naturally derived and/or of high natural origin content, having a high percent modern carbon content (pMC), such as bio-based ethylene, chemicals derived therefrom as described herein, and polymers (e.g., polyethylene) and copolymers, such as EVA from biomass sources, such as from bio-based ethylene, such as from bio-based ethanol. The ethanol can be produced from biomass—for example, by fermenting sugars (from a biomass source, e.g., a starchy material, a cellulosic material, a lignocellulosic material or mixtures of these) into ethanol. The bio-based ethanol (e.g., cellulosic ethanol) is dehydrated, for example, using a solid catalyst in a continuous flow reactor, to produce bio-based ethylene gas in high yield. This bio-based ethylene can be used to manufacture polymers, such as polyethylene (PE), polyethylene terephthalate (PET), polyvinyl chloride (PVC), and polystyrene (PS), as well as fibers and other organic chemicals, such as those depicted in FIGS. 1-7. Blends of any biomass-derived material, such as any monomeric or polymeric material described herein, and a fossil-fuel derived material, such as a fossil fuel-derived equivalent of the biomass-derived material utilized in any composition, such as blends of fossil fuel-derived caprylyl glycol and biomass-derived caprylyl glycol, can be produced if desired, for example, as a method of providing a favorable balance between cost and GWP reduction and natural-origin and high modern carbon content, which is an important attribute for the consumer of today.

The terms “sustainably,” “sustainability,” and “sustainable” are used herein to describe processes by which organic compounds are produced from carbon-based raw materials that are obtained from renewable carbon sources. More particularly, “sustainable” processes utilize raw materials that are obtained from carbon sources other than fossil fuels. In this context, a “renewable” carbon source is an aboveground, recently living source of carbon, such as biomass. The carbon from “renewable” carbon sources may be fixed through photosynthetic processes. Because the “sustainable” methods disclosed herein do not utilize, or only use them to a certain degree and, preferably, only to a limited degree, subterranean carbon sources (e.g., fossil fuels), as a source for carbon-based raw materials in the disclosed syntheses, such “sustainable” methods have the potential to drastically reduce or practically eliminate the amount of net new carbon introduced into the earth's atmosphere and oceans as compared to the conventional production processes of the compounds discussed herein. This is so because the carbon-based starting materials used in these sustainable products are obtained from carbon sources that are already above the earth's surface (renewable sources such as biomass) rather than from carbon sources that must be extracted from a subterranean environment, such as fossil fuels. Sustainability, for example in making cosmetics, can be achieved by using natural, naturally derived, natural-origin content as described in ISO 16128-1, First Edition (2016 Feb. 15) and ISO 1628-2, First Edition (2017 September), which are hereby incorporated by reference herein in their entirety.

The term “bio-based” and prefix “bio-” are used herein to describe compounds and materials (e.g., bio-based ethylene, bio-based polyethylene, bio-α-olefins) that are made, at least in part, from renewable raw materials (e.g., biomass) and that therefore are at least partially made of modern carbon. In certain embodiments, at least about 50% and up to about 100% of the carbon in such bio-based compounds and materials is modern carbon. The term “bio-based” can also be used to describe carbon content (% bio-based carbon or pMC), to distinguish carbon obtained from recent living matter (e.g., biomass) from carbon obtained from fossil fuels.

The carbon found in and obtained from biomass has a different radiocarbon (Carbon-14 or C14) signature compared to carbon found in and obtained from fossil fuels. Atmospheric carbon contains a small but measurable fraction of Carbon-14, which is processed by green plants to make organic molecules during photosynthesis. Thus, the fraction of Carbon-14 in organic molecules in biomass reflects the fraction of Carbon-14 currently in the atmosphere. In contrast, the organic molecules in fossil fuels contain no Carbon-14, or very little.

The percentage of bio-based carbon in a sample can be determined by Carbon-14 analysis, and a standardized methodology for Carbon-14 analysis is described in ASTM D6866-18, the entire contents of which is hereby incorporated by reference herein.

In certain embodiments of the present invention, the percentage of bio-based carbon in bio-based ethylene, and in bio-based polymers and other materials made from the bio-based ethylene, is about 80% or more, about 85% or more, about 90% or more, about 95% or more, or about 100%. In particular embodiments, the carbon in bio-based ethylene, and in bio-based polymers and other materials made from the bio-based ethylene, is about 100% bio-based carbon.

The percent modern carbon (pMC) describes the ratio of the amount of radiocarbon (Carbon-14) in a sample to the amount of radiocarbon in a modern reference standard. A modern reference standard commonly used is a National Institute of Standards and Technology standard (SRM 4990C) with a radiocarbon content approximately equivalent to the fraction of atmospheric radiocarbon in the year 1950 AD. The amount of radiocarbon in the modern reference standard represents 100 pMC. Because fossil fuels do not contain Carbon-14, a sample having carbon that is only petroleum-based carbon, for example, would have approximately 0 or 0 pMC. Further, because the fraction of Carbon-14 in the atmosphere today is higher than it was in 1950 AD, the pMC of materials made from recent biomass (e.g., biomass from sources living in the past 2-5 years) may be higher than 100 pMC. A number of certified testing labs are available to do this testing, including Beta Analytic Inc., 4985 SW 74th Court, Miami, Fla. 33155.

In certain embodiments of the present invention, the carbon in bio-based ethylene, and in bio-based polymers, copolymers, polymeric blends, for example, melt blended intimate polymer blends of 100 pMC polymers and 0 pMC polymers, and other materials made from the bio-based ethylene or any bio-based material described herein, is at least about 80 pMC, at least about 85 pMC, at least about 90 pMC, at least about 95 pMC, at least about 99 pMC, or about 100 pMC.

Ethylene and other ethylene-based compounds described herein (e.g., 1,2-diols and polymers and copolymers of ethylene) are conventionally heretofore produced through the processing of petroleum. As a result, the production of these products often requires the consumption of fossil fuels, and consumer demand for products made from ethylene and other ethylene-based compounds, coupled with increasing population, will drive increased extraction and consumption of fossil fuels, which is a non-renewable and precious resource. Among other things, the processing and consumption of fossil fuels increases the amount of carbon dioxide in the atmosphere and hydrocarbons that escape processing, many of which are more powerful greenhouse gases, such as methane. An increase in the amount of atmospheric carbon dioxide can contribute to long-term climate change and global warming through the “greenhouse effect,” wherein gases such as carbon dioxide prevent a portion of the sun's radiated energy from escaping the earth's atmosphere. Atmospheric carbon dioxide levels have increased over thirty percent since 1958, largely driven by the increased usage of fossil fuels, driven by increasing demand for transportation fuels and electricity.

Using bio-based materials, such as bio-based ethanol obtained from biomass to sustainably produce bio-based ethylene and other products (e.g., polymers and copolymers of bio-based ethylene) can reduce the consumption of fossil fuels by providing an alternative and environmentally friendly source of ethylene that does not require fossil fuels as a source material. Using such bio-based materials, such as bio-based ethylene to sustainably make widely used polymers such as, e.g., polyethylene, can reduce the net increase of carbon in the atmosphere and oceans. A convenient source of aromatic compounds that can be useful in producing high natural origin compounds, as described herein, is wood-tar creosote, not to be confused with coal-tar creosote which contain polycyclic aromatic hydrocarbons (“PAH”). Wood-tar creosote can be produced from pyrolysis of, for example, beech, oak or pine wood, in the absence of oxygen, producing as products charcoal and wood-tar creosote, which is a mixture of monophenols, guaiacol, creosols and homologs. For example, the fragrant, yellow wood-tar creosote contains varying amounts of phenol, o-cresol, m- and p-cresols, o-ethylphenol, guaiacol, 3,4-xylenol and 3,5-xylenol. Such compounds can be ‘upgraded’ by various reactions described herein, including catalytically upgraded to a desired aromatic compound. For example, as described herein, guaiacol can be catalytically de-methoxylated to produce 100 pMC bio-based phenol, which can be utilized to produce many compounds described herein, including phenoxyethanol, and other preservatives. Another source of aromatic compounds are those described in U.S. Pat. No. 10,597,595, the entire contents of which is hereby incorporated by reference herein.

As a further example, bio-based ethylene and other bio-based materials described herein may be utilized to produce bio-based 1,2-alkyldiols (e.g., 1,2-octanediol) and bio-based phenoxyethanol, for example, produced by the reaction of bio-based phenol, for example, produced by de-methoxylation of guaiacol, with bio-based ethylene oxide or bio-based 2-chloroethanol, such as 100 pMC 2-chloroethanol. Bio-based 1,2-alkyldiols may be used alone or in combination with other preservatives to produce cosmetic preservative products for use in cosmetics, such as creams, jellies, ointments, pastes, cerates, chrisms, cosmetics, demulcents, emulsions, essences, liniments, salves, unctions, unguents, or moisturizers wherein said bio-based 1,2-alkyldiols contain four, six, eight, ten, twelve or more carbons per bio-based 1,2-alkyldiol, and wherein the percent modern carbon (pMC) of the preservative system and/or cosmetic overall is, for example, at least about 50 pMC, such as at least about 60, 70, 80, 90 percent or more, e.g., at least about 91, 92, 93, 94, 95, 96, 97, 98, 99 or more percent, or such as at least about 100 pMC. These aforementioned cosmetic products may further comprise bio-based phenoxyethanol as part of the preservative system. Phenoxyethanol is commonly employed as a preservative in, for example, cosmetic products, and phenoxyethanol is conventionally heretofore produced from ancient carbon sources, such as fossil fuels. Therefore, the use of bio-based phenoxyethanol as a preservative or additive in these cosmetic products has the effect of further substantially reducing or completely eliminating the use of non-sustainable ingredients in these cosmetic products. As a result, it is possible to sustainably produce a cosmetic product with a percent modern carbon of at least about 50 pMC, such as at least about 60, 70, 80, 90 percent or more, e.g., at least about 91, 92, 93, 94, 95, 96, 97, 98, 99 or more percent, or such as about 100 pMC according to embodiments of the invention.

Production of Bio-Based Ethylene from Bio-Based Ethanol

The present invention provides materials and processes, including catalysts and reaction conditions, useful in converting bio-based ethanol into bio-based ethylene. Because ethanol can be made from biomass, the ability to convert ethanol to ethylene allows for the production of bio-based ethylene from various biomass sources (e.g., agricultural waste, which can be used to produce cellulosic ethanol, which then can be converted to bio-based ethylene). Catalysts useful in converting bio-based ethanol into bio-based ethylene include but are not limited to metal oxides, silico-aluminates, silico-aluminophosphates, and heteropoly acids.

### Catalyst One

Metal oxides, including transition metal oxides, that can be used as catalysts to convert bio-based ethanol into bio-based ethylene include, for example, Al₂O₃, TiO₂—Al₂O₃, SiO₂, SiO₂—Al₂O₃, ZrO₂, WO₃, ZnO/Al₂O₃, and MgO—Al₂O₃/SiO₂. Aluminum oxide (Al₂O₃) includes, for example γAl₂O₃ or calcined (or a) Al₂O₃. In addition, zeolites such as ultrastable Y (USY) zeolites can be added to metal oxides, such as Al₂O₃, to provide suitable acidity.

Silico-aluminates useful as catalysts for converting bio-based ethanol into bio-based ethylene include, for example, zeolites with varying Si/Al ratios, as well as sodium- and phosphorous-modified zeolites with varying of phosphorous loading, such as 1 to 20 percent by weight. Such zeolites include USY and ZSM5, for example. In certain embodiments, the Si/Al ratio ranges from 20-360 (e.g., 20 silica to 1 aluminum, up to and including 360 silica to 1 aluminum). For example, the Si/Al ratio can be greater the about 18, 19, 20, or more, such as greater than about 20, 22, 28, 32, 36, 40, or more, such as greater than about 50, 60, 70, 80, 90, 100, 150, or more, such as greater than about 200, 250, 300 or more, such as greater than about 350. In other embodiments, the Si/Al ratio is between about 18 and 360, such as between about 20 and 300, between about 22 and 250, between about 24 and 200, between about 30 and 170 or between about 35 and 150.

Silico-aluminophosphates useful as catalysts for converting bio-based ethanol to bio-based ethylene include, for example, zeolites treated with H₃PO₄. Such zeolites may be prepared by using a simple impregnation method followed by drying and calcination, as described herein.

Heteropoly acids useful as catalysts for converting bio-based ethanol to bio-based ethylene include, for example, molybdophosphoric acid and tungstophosphoric acid.

In certain embodiments, a catalyst may be modified with a metal (such as a transition metal, for example lanthanum) to improve the activity and stability of the catalyst. In some embodiments, the catalyst is modified to contain between about 0.05 and about 15 percent by weight of one or more lanthanide elements, such as between about 0.05 and about 10 percent by weight, between about 0.1 and about 7.5 percent by weight or between about 0.5 and about 5 percent by weight of a lanthanide element (elements 57 through 71, inclusive). In particular embodiments, the lanthanide element is lanthanum, and the catalyst includes between about 0.5 and about 5 percent by weight lanthanum, such as between about 0.7 and about 2.5 percent by weight, or between about 0.8 and about 1.5 percent by weight lanthanum. In a specific embodiment, the catalyst contains about 1% by weight lanthanum. In a particular embodiment, the catalyst may be modified to contain, for example, 1-5% lanthanum. Other metals that may be used include hafnium, cerium, praseodymium, neodymium, samarium, and other large radii metals having an atomic radius of greater than about 150 pm. Alternatively, a catalyst may be modified with phosphorous or a base. Bases that can be used to modify the catalyst include, for example, strong bases, such as sodium hydroxide, potassium hydroxide, calcium hydroxide, and ammonium hydroxide, and various weak bases, such as nitrogen bases, e.g., ammonia or pyridine.

A catalyst reaction as provided herein may be described in terms of activity, or the percentage of bio-based ethanol that is converted to other compounds (including bio-based ethylene). In certain embodiments, a catalyst reaction as described herein achieves 80-100% ethanol conversion—that is, 80-100% by weight of the bio-based ethanol is converted to other compounds (including bio-based ethylene) during reaction with the catalyst. In some embodiments, 90-100% by weight of the ethanol is converted, and in further embodiments, 95-100% by weight of the ethanol is converted. In other embodiments, the catalyst reaction achieves 90-98% by weight ethanol conversion.

In further embodiments, quantitative conversion of ethanol reduces or eliminates ethanol breakthrough; reducing or eliminating such breakthrough is often desirable, as ethanol contamination in the ethylene gas can influence downstream processes, such as by poisoning catalysts used in downstream polymerization processes. In specific embodiments, conversion of ethanol is greater than 99 percent by weight, such as greater than 99.5, 99.6, 99.8, 99.9 or 99.95 percent by weight.

A catalyst reaction as provided herein may also be described in terms of selectivity, or the ability to convert bio-based ethanol to ethylene specifically. The relative formation of ethylene can be expressed in terms of molar ethylene selectivity, or the mole percent of ethylene produced. For example, in some embodiments, at least about 50 mole percent of ethylene is produced. In further embodiments, at least about 60 mole percent of ethylene, at least about 70 mole percent of ethylene, at least about 80 mole percent of ethylene, at least about 90 mole percent of ethylene, at least about 95 mole percent of ethylene, at least about 98 mole percent of ethylene, or about 100 mole percent of ethylene is produced.

In some embodiments, bio-based ethanol is dehydrated in the vapor phase, inside a fixed-bed or fluidized-bed reactor containing the catalyst. Liquid bio-based ethanol is added to the catalyst bed at a specified flow rate, and is vaporized. Several parameters, including temperature and liquid bio-based ethanol flow rate, may influence activity and/or selectivity of a catalyst reaction. For example, a rise in temperature through the catalyst bed can lead to carbon deposits (‘coke’) on the catalyst; because such deposits can reduce ethylene yield, it may be desirable to control the temperature through the catalyst bed to avoid or reduce the formation of such deposits. In some embodiments, after 24 hours of running the catalytic dehydration reaction at 400° C., the catalyst contains 1 percent by weight or less bound carbon (‘coke’), such as 0.75 percent by weight or less, 0.5 percent by weight or less, 0.25 percent by weight or less (e.g., 0.1 percent by weight or less) bound carbon (‘coke’).

In certain embodiments, the catalyst reaction converting bio-based ethanol into ethylene is performed at a temperature that is within the range of 250-500° C. In some embodiments, the catalyst reaction is conducted at a temperature that is within the range of 325-425° C., for example, at a temperature of 400° C. In other embodiments, the catalyst reaction is conducted at a temperature that is below 300° C.

In certain embodiments, the flow rate of liquid bio-based ethanol into the catalyst bed is within the range of 0.2-0.5 mL/min. In further embodiments, the bio-based ethanol flow rate is 0.25 mL/min; in other embodiments, the bio-based ethanol flow rate is 0.3 mL/min; in still other embodiments, the bio-based ethanol flow rate is 0.4 mL/min. While higher flow rates may be appropriate for larger catalyst beds, a bio-based ethanol flow rate that is too high may cause bio-based ethanol breakthrough into the ethylene product.

In certain embodiments, the bio-based ethanol used in the reactions described herein contains a diluent. For example, in some embodiments, the bio-based ethanol contains from about 0.1 to about 50 percent diluent by weight. In further embodiments, the bio-based ethanol contains between about 0.5 and about 25 percent by weight diluent, or between about 1 and about 10 percent by weight diluent. In certain embodiments, the bio-based ethanol contains about 8 percent by weight diluent. A preferred diluent is water. In some embodiments, a diluent (for example, water) acts as a cooling agent or a washing agent for the reactor, which reduces ‘coke’ formation.

The volume of catalyst used in the reaction depends in part on the size of the reactor, e.g., as reflected by the diameter of a reaction column. For example, for a diameter of 1 cm, the volume of catalyst may be 3-12 cc, e.g., 11 cc; a bio-based ethanol flow rate used for such a reaction column may be, e.g., 0.3 mL/min. For a diameter of 2 cm, the volume of catalyst may be 44 cc; and a bio-based ethanol flow rate used for such a reaction column may be, e.g., 1.2 mL/min. For a diameter of 3 cm, the volume of catalyst may be 99 cc; and a bio-based ethanol flow rate used for such a reaction column may be, e.g., 2.7 mL/min. For a diameter of 4 cm, the volume of catalyst may be 176 cc; and a bio-based ethanol flow rate used for such a reaction column may be, e.g., 4.8 mL/min. For a diameter of 6 cm, the volume of catalyst may be 396 cc; and a bio-based ethanol flow rate used for such a reaction column may be, e.g., 10.8 mL/min.

The rate at which bio-based ethanol in liquid form is introduced into the reactor may also be expressed in terms of liquid hourly space velocity (“LHSV”), which is the ratio of liquid volume per hour divided by volume of catalyst. Because it is a ratio, LHSV can be used to generally describe a liquid flow into a reactor regardless of reactor volume, and LHSV is a measure that may aid in scaling bench-scale processes to mass production processes. In certain embodiments, the LHSV of bio-based ethanol in liquid form may be between about 0.5 h⁻¹ and about 20 h⁻¹, such as between about 1 h⁻¹ and about 15 h⁻¹, between about 1.2 h⁻¹ and about 10 h⁻¹, or between about 1.5 h⁻¹ and about 7.5 h⁻¹.

In certain embodiments, the LHSV of bio-based ethanol in liquid form may be between about 1 to about 2.5 h⁻¹, such as between about 1.25 h⁻¹ and about 2.25 h⁻¹, such as between about 1.5 h⁻¹ and 2.0 h⁻¹. In further embodiments, the LHSV of bio-based ethanol in liquid form may be about 1.25 h⁻¹, in other embodiments, the LHSV of bio-based ethanol in liquid form may be about 1.5 h⁻¹, in still other embodiments, the LHSV of bio-based ethanol in liquid form may be about 2.0 h⁻¹. While greater LHSVs may be appropriate for larger catalyst beds, an LHSV of bio-based ethanol that is too great may cause bio-based ethanol breakthrough into the ethylene product.

Gaseous by-products of producing bio-based ethylene from bio-based ethanol include carbon dioxide and carbon monoxide, both being good ligands and, as a result, capable of poisoning catalytic activity or altering its reaction course and products produced. In some embodiments, the amount of carbon dioxide or carbon monoxide in the bio-based ethylene produced from conversion of bio-based ethanol to bio-based ethylene is less than 1000 ppm by weight, such as less than 750 ppm by weight, less than 600 ppm by weight, less than 500 ppm by weight, less than 250 ppm by weight, less than 100 ppm by weight, less than 50 ppm by weight, or less than 10 ppm by weight. In some instances, the concentration of carbon dioxide and the concentration of carbon monoxide are each less than 10 ppm by weight, such as less than 5 ppm by weight, less than 3 ppm by weight, or less than 1 ppm by weight. If desired, scrubbers can be used to remove carbon dioxide or carbon monoxide. In addition, zeolites or molecular sieves may be used to remove carbon monoxide or carbon dioxide or unreacted bio-based ethanol. In some embodiments, the concentration of bio-based ethanol in the bio-based ethylene that is produced is less than 100 ppm by weight, less than 50 ppm by weight, or less than 10 ppm by weight, such as, e.g., less than 5 ppm by weight or less than 1 ppm by weight. In particular embodiments, carbon dioxide is scrubbed from ethylene produced by utilizing a column packed with molecular sieves −5 A, molecular sieves −3 A and carbolime. In particular embodiments carbon monoxide is scrubbed from ethylene produced by utilizing a column packed with Cu(I) dispersed on activated carbon or zeolites.

Ethanol produced from any source can be used to make ethylene and polymers (e.g., polyethylene) using the catalysts described herein. In certain embodiments, the ethanol is produced from biomass. Systems and methods for producing ethanol from biomass are described in U.S. Pat. Nos. 9,644,244, 9,677,039, 9,708,761, and 9,816,231, the disclosures of which are incorporated by reference herein in their entireties. In certain preferred embodiments, the biomass used to make ethanol is non-food biomass (e.g., agricultural waste).

### Uses of Bio-Based Ethylene

Bio-based ethylene can be used in place of fossil fuel-based ethylene for various applications and to produce a variety of end products. For example, bio-based ethylene can be oxidized to form ethylene oxide, which is used, for example, to produce surfactants and detergents. The bio-based ethylene oxide obtained from the oxidation of bio-based ethylene may be further reacted with phenol, such as 100 pMC phenol, e.g., from de-methoxylation of guaiacol, to obtain bio-based phenoxyethanol. As another example, the bio-based ethylene may be reacted with a hypohalous acid, or an equivalent, such as a trihaloisocyaruric acid, such as trichloroisocyanuric acid, to form a natural-origin, bio-based halohydrin of high pMC content. For example, the bio-based ethylene may be reacted with hypochlorous acid (HOCl) to form bio-based 2-chloroethanol. The bio-based chloro-ethanol may then be further reacted with a phenoxide ion source to obtain bio-based phenoxyethanol. Bio-based ethylene can also alkylate benzene to ethylbenzene, which is used to produce styrene and polystyrene. The chemicals and other products made from bio-based ethylene in turn will be bio-based. For example, ethylene can be produced from ethanol, e.g., bio-based ethanol, and benzene can be catalytically alkylated with the bio-based ethylene, and then the resulting ethylbenzene can be dehydrogenated to produce styrene, which can be, for example, polymerized alone or in combination with other monomers, such as one or more bio-based monomers, such as ethylene to produce copolymers of styrene, such as a POE of styrene that is bio-based, such as one that is greater than about 25 pMC, such as greater than about 35, 45, 55, 65, 75, 85 pMC or more, such as greater than 95 pMC, such as greater than about 96, 97, 98 or more pMC, such as greater than about 99.5 pMC, or about 100 pMC.

In certain embodiments, the bio-based ethylene as described herein can be used to produce different polymers, including different grades of polyethylene; using bio-based ethylene to produce such polymers generates bio-based polymers (e.g., bio-based polyethylene, bio-based polyethylene terephthalate (PET)). For example, PET is a condensation polymer of ethylene glycol and terephthalic acid; the ethylene glycol portion of the polymer can be made from bio-based ethylene, by forming ethylene oxide and reacting the ethylene oxide with water. For example, a bio-based PET can be produced by producing bio-based ethylene and converting the bio-based ethylene to bio-based ethylene oxide; ring opening the ethylene oxide, for example, with, acid, water or a base, such as hydroxide, to produce ethylene glycol that is of natural origin. The natural-origin ethylene glycol can be reacted with a terephthalic acid, such as one or more of o-, m- or p-terephthalic acid, or an ester thereof, to produce a bio-based PET.

Methods of producing polyethylene are described, for example, in U.S. Pat. Nos. 4,530,914, 5,132,380, 6,063,879, 6,221,985, 6,277,931, and 7,650,930, which are incorporated by reference in their entireties. Methods of producing PET are provided in U.S. Pat. Nos. 2,534,028, 5,898,059, and 7,015,267, which are incorporated by reference herein in their entireties.

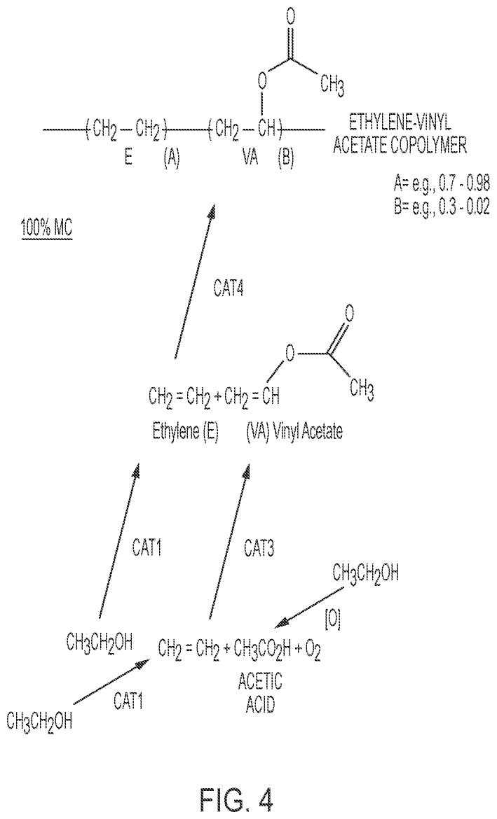

In some embodiments, the bio-based ethylene is copolymerized with other monomers, such as, for example, propylene or vinyl acetate to produce bio-based ethylene copolymers, such as bio-based ethylene-vinyl acetate (EVA) copolymers. An exemplary reaction schematic for the production of bio-based EVA copolymers is provided in FIG. 4 and described in further detail herein. In other embodiments, the bio-based ethylene is polymerized to produce bio-based polyethylene, and the bio-based polyethylene is modified to produce, for example bio-based chlorinated or chlorosulfonated polyethylene. Propylene can be produced from ethylene utilizing metathesis technology developed by ABB Lummus Global Olefins Conversion Technology (OCT) and technology developed by Phillips Petroleum. For example, an ethylene-propylene polymer can be produced by making bio-ethylene using olefin metathesis to produce propylene. The copolymer is produced by reacting bio-based ethylene and propylene.

Bio-based polyethylene produced according to the methods described herein includes, for example, low density bio-based polyethylene, medium density bio-based polyethylene, high density bio-based polyethylene and linear low density polyethylene, such as produced by reacting one or more bio-based alpha olefins as described herein, such as one or more of a C4-C10 bio-α-olefins, such as 1-octene with bio-ethylene to produce a bio-linear low density polyethylene. Such bio-based polyethylene of the different densities can be used in a variety of applications, such as, e.g., consumer goods including single-use water bottle closures and carton overwraps. Such a bio-based polyethylene can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

### Other Uses and Processes of Bio-Based Ethylene

Bio-based ethylene can be utilized in all those current processes as ethylene derived from ancient carbon sources, such as fossil fuels. Major processes and other uses include oxidation, for example, to produce ethylene oxide, halogenation, for example, to produce ethylene dichloride, hydrohalogenation, alkylation, arylation, for example, to produce ethylbenzene, for example, for the production of polystyrene, hydration, oligomerization, hydroformylation, for example, to produce aldehydes and ketones, and addition of a hypohalous acid to form a halohydrin. Other reactions for bio-based ethylene include, for example, Heck coupling, hydroalkenylation, oxymercuration, Büchi reaction, cyplopropanation, hydrophosphination, Diels-Alder reaction, hydroboration, and hydroacylation. There has been a long-felt need to produce such products from naturally derived, high natural-origin materials, but such products have proved elusive heretofore. Embodiments elucidated herein open an entire world of in-demand, high natural-origin, high modern carbon content, bio-based, and sustainable materials and such embodiments place biomass feedstock on an equal footing with fossil fuel-derived analogues, which allows for the sustainable production of high-percentage modern carbon everyday materials.

Oligomerization of bio-ethylene allows for the formation of bio-α-olefins having an even number of carbon atoms, for example, terminal or bio-α-olefins having C2n number of carbon atoms, where n is a positive integer between, for example, 1 and about 1000, such as between 2 and about 100, or between 1 and about 50 or such as between 1 and about 25. For example, the bio-α-olefins can have 2, 4, 6, 8, 10, 12, 14, 16, 18 or 20 carbon atoms, or the bio-α-olefins can be mixtures of these bio-α-olefins, such as mixtures of 2, 4, 6, 8, and 10 number of carbon atoms. The α-olefins can be mixtures of bio-α-olefins and fossil fuel-derived α-olefins to balance the cost, GWP and natural-origin content. Olefin metathesis, for example, can be utilized to provide bio-based molecules having odd numbers of carbon atoms, for example, 3, 5, 7, 9 or 11 carbon atoms. Another approach, for example, for making odd number of carbon atoms would be to make a halogenated molecule or molecules from an ethylene oligomer or oligomers having an even number of carbon atoms and then reacting that halogen with a carbon nucleophile, such as a Grignard reagent, an alkyl zinc compound, an alkyl copper compound or an alkyl lithium reagent having an odd number of carbons to produce a molecule having an odd number of carbon atoms.

### Oligomerization of Ethylene to Alpha Olefins

Ethylene can be oligomerized using a number of commercial processes, including the Chevron Phillips Process, the Ineo Process, Shell's Process (Shell Higher Olefin Process or SHOP process), the Idemitsu's Process, the Vista Alfene Process, Exxon's Process, Dupont's Versipol Process, Sabic/Linde process and the Sasol process. Suitable processes are described in J. Jiang et al., Appl. Petrochem Res., Vol. 6, no. 4 (2016), pp. 413-17; A. Bollmann et al., J. Am. Chem. Soc., Vol. 126, no. 45 (2004), pp. 14712-13; G. P. Belov et al., Petroleum Chem., Vol. 52, no. 3 (2012), pp. 139-54; and in U.S. Pat. Nos. 4,409,414, 7,297,832, 4,434,312, 4,783,573, 4,628,138, and 7,300,904, the disclosure of each of which is hereby incorporated by reference herein in its entirety.

### Reactions of Alpha Olefins

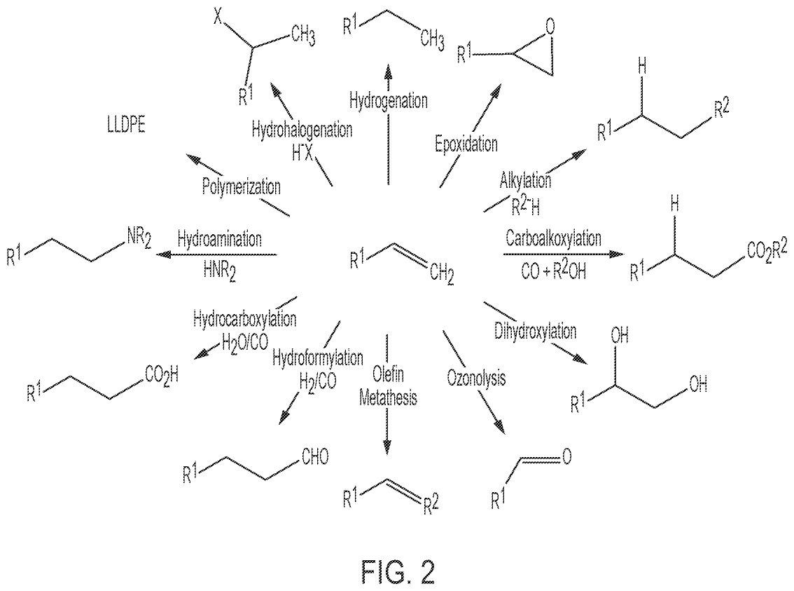

Referring now to FIG. 2, many reactions and molecules are possible from α-olefins and/or ethylene provided and derived, at least in part, from biomass. For example, as described herein, a bio-α-olefin (and/or bio-based ethylene) can be hydrogenated to produce an alkane, for example, a straight chain alkane; a bio-α-olefin (and/or bio-based ethylene) can be epoxidized to produce an epoxide; a bio-α-olefin (and/or ethylene) can be alkylated to produce a higher alkane, for example, a straight chain alkane; a bio-α-olefin (and/or bio-based ethylene) can be carboalkoxylated to produce a ester; a bio-α-olefin (and/or bio-based ethylene) can be dihydroxylated to produce a 1,2-alkyldiol; a bio-α-olefin (and/or bio-based ethylene) can be ozonated to produce aldehyde; a bio-α-olefin (and/or bio-based ethylene) can undergo olefin metathesis to produce another bio-α-olefin; a bio-α-olefin (and/or bio-based ethylene) can undergo a hydroformylation reaction to produce an aldehyde; a bio-α-olefin (and/or bio-based ethylene) can undergo a hydrocarboxylation to produce an organic acid; a bio-α-olefin (and/or bio-based ethylene) can undergo a hydroamination to produce an amine, such as a primary, secondary or tertiary amine; a bio-α-olefin (and/or bio-based ethylene) can undergo polymerization alone, or in combination with another monomer, such as ethylene, to produce a polymer; or a bio-α-olefin (and/or bio-based ethylene) can be hydrohalogenated to produce a halogenated alkane. The halogen can add in a Markovnikov or in an anti-Markovnikov sense. Such natural-origin, high modern carbon content bio-molecules produced by these reactions and methods can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-α-olefins (and/or bio-based ethylene), or blends of such with analogous fossil fuel-derived α-olefins (and/or ethylene), can be hydrogenated, for example to alkanes utilizing homogeneous hydrogenation catalysts, for example, by using Wilkinson's catalyst and hydrogen or a heterogeneous catalyst, for example, a metal, such as nickel or a precious metal, such as platinum or platinum on a support. The resulting hydrogenated compositions can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-α-olefins (and/or bio-based ethylene), or blends of such with analogous fossil fuel-derived α-olefins (and/or ethylene), can be epoxidized, for example, using hydrogen peroxide, sodium periodate, t-butyl hydroperoxide, peroxyacids, such as m-CPBA or mixtures of these alone or in combination with a metal catalyst, such as ruthenium chloride or osmium tetroxide. Chiral epoxides can often be derived enantioselectively from prochiral alkenes. Many metal complexes give active catalysts, for example those including titanium, vanadium, and molybdenum. The resulting epoxidized compositions can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-α-olefins (and/or bio-based ethylene), or blends of such with analogous fossil fuel-derived α-olefins (and/or ethylene), can be alkylated to alkanes, such as branched alkanes using, for example, an acid, e.g., HF or sulfuric acid. The resulting alkane compositions can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-α-olefins (and/or bio-based ethylene), or blends of such with analogous fossil fuel-derived α-olefins (and/or ethylene) can be carboalkoxylated, for example, using carbon monoxide and an alcohol, to make corresponding ester. The transformation can be completed utilizing a palladium catalyst, such as Pd[C₆H₄(CH₂PBu-t)₂]₂. The resulting carboxylated compositions can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Still referring to FIG. 2, and as discussed herein, bio-α-olefins (and/or bio-based ethylene), or blends of such with analogous fossil fuel-derived α-olefins (and/or ethylene), can be dihydroxylated to the corresponding 1,2-diols, such as 1,2-dihydroxyhexane or 1,2-dihydroxyoctane (caprylyl glycol). Such a transformation can be completed utilizing a number of reagents, including sodium periodate with ruthenium trichloride, osmium tetroxide and water, asymmetric ligands, along with hydrogen peroxide and osmium tetroxide (Sharpless reaction). Other reagents include Milas reagent, Upjohn dihydroxylation, and the Prevost and Woodward dihydroxylation. The resulting dihydroxylated compositions can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-alpha-olefins and/or bio-ethylene, or blends of such with analogous fossil fuel-derived alpha olefins and/or ethylene can be lysed with ozone to aldehydes including one less carbon atom than the starting bio-α-olefin and/or bio-ethylene, which in turn can be converted to many different compounds, including alcohols by reduction. The resulting lysed compositions can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Other olefins can be produced from bio-α-olefins (and/or bio-based ethylene), or blends of such with analogous fossil fuel-derived α-olefins (and/or ethylene), e.g., cross metathesis or self-metathesis. Typical catalysts include, for example, Schrock's 1-Mo, Grubbs 2-Ru, 3-Ru or 4-Ru catalysts. For example, styrene can be metathesized to 1-phenyl-1-octene using 1-Mo, as described in Crowe, W. E, J. Am. Chem. Soc., Vol. 115, no. 23 (1993), pp. 10998-99, the disclosure of which is hereby incorporated by reference herein in its entirety. Other metathesis reactions are described in Andrew G. Myers Research Group, Chemistry 115 Handouts, “The Olefin Metathesis Reaction”, pp. 1-38 (2019), the entire contents of which is hereby incorporated by reference in its entirety. Such other bio-olefins can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-α-olefins (and/or bio-based ethylene), or blends of such with analogous fossil fuel-derived α-olefins (and/or ethylene) can be hydroformylated using carbon monoxide and hydrogen, along with a catalyst to produce the corresponding aldehyde. Catalysts can include, for example, cobalt or rhodium. Specific examples include tris(triphenylphosphine)rhodium carbonyl hydride, and cobalt tetracarbonyl hydride. Such bio-based high natural-origin and modern carbon content hydroformylated materials can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Carboxylic acids can be prepared from bio-α-olefins (and/or bio-based ethylene), or blends of such with analogous fossil fuel-derived α-olefins (and/or ethylene) described herein via hydrocarboxylation using hydrogen, carbon dioxide and a catalyst. For example, a suitable catalyst is a combination of [RhCl(cod)]2 (cod=cyclooctadiene) as a catalyst and diethylzinc as a hydride source. Such bio-based acids can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Still referring to FIG. 2, bio-α-olefins (and/or bio-based ethylene), or blends of such with analogous fossil fuel-derived α-olefins (and/or ethylene), described herein can be hydroaminated to corresponding amines, for example using a strong base, or group IV catalysts, such as titanium or zirconium catalysts. Such bio-based amines can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Bio-α-olefins (and/or bio-based ethylene), or blends of such with analogous fossil fuel-derived α-olefins (and/or ethylene), can be useful in creating polyethylenes with special properties, such as low density and/or elastomeric properties. For example any of the α-olefins described herein, such as bio-α-olefins, such as bio-1-octene from dehydration of ethanol, followed by tetramerization, can be copolymerized with ethylene from ethanol to produce a linear low density elastomer (LLDPE) that is 100 pMC. In other embodiments, such bio-based polymers and copolymers can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Still referring to FIG. 2, bio-α-olefins (and/or bio-based ethylene), or blends of such with analogous fossil fuel-derived α-olefins (and/or ethylene), may be hydrohalogenated, for example, in a Markovnikov or anti-Markovnikov manner. For example, Markovnikov addition can be completed using a strong acid, such as hydrochloric or hydrobromic acid and anti-Markovnikov addition can be completed using, for example, HBr and a radical source, such as a peroxide. The resulting high natural-origin and modern carbon content bio-based hydrohalogenated compounds and materials can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

The reactions schematically depicted in FIG. 2 may also utilize bio-based ethylene as a starting material. For example, and without limitation, bio-based ethylene may be oxidized in the presence of oxygen and a catalyst (e.g., a silver-based catalyst) to form bio-based ethylene oxide. Other suitable catalysts for the oxidation of ethylene to ethylene oxide are known in the art and would likewise be suitable for the oxidation of bio-based ethylene of the invention. Bio-based ethylene oxide can then be further reacted with phenolic compounds, for example, bio-based phenol, in the presence of, for example, an alkali-metal hydroxide to produce bio-based phenoxyethanol.

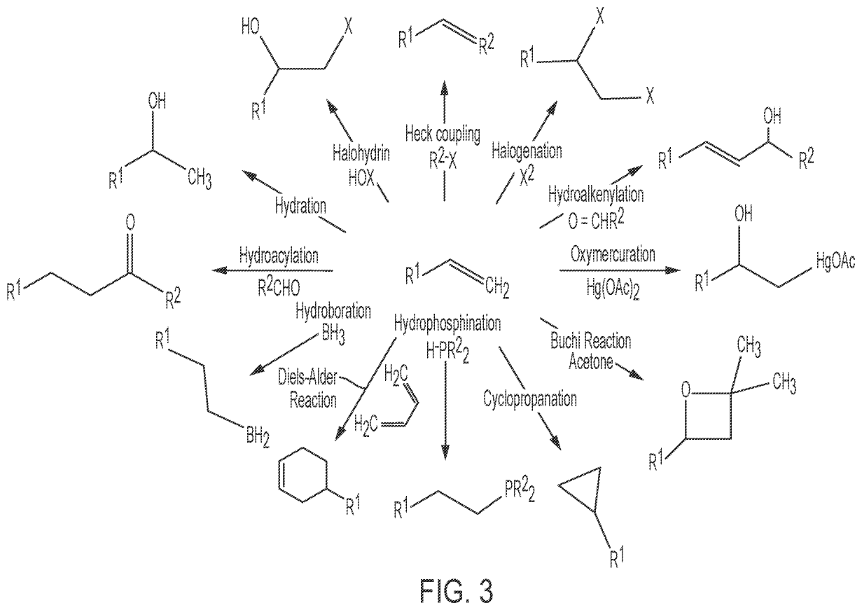

Continuing now by referring to FIG. 3, many other reactions and molecules are possible from α-olefins and/or ethylene provided, and derived, at least in part, from biomass. For example, as described herein, a bio-α-olefin (and/or ethylene) can be undergo a Heck coupling to produce an internal bio-olefin; a bio-α-olefin (and/or bio-based ethylene) can be halogenated, such as with chlorine or bromine to produce an di-halogenated bio-material; a bio-α-olefin (and/or bio-based ethylene) can be hydroalkenylated to produce a higher alkene; a bio-α-olefin (and/or bio-based ethylene) can be oxymercurated to add an acetoxymercury (HgOAc) group and a hydroxy (OH) group across the double bond; a bio-α-olefin (and/or bio-based ethylene) can undergo a Büchi reaction with acetone to produce a 4-membered ring system; a bio-α-olefin (and/or bio-based ethylene) can be cyclopropanated to produce 3-membered ring system; a bio-α-olefin (and/or bio-based ethylene) can undergo hydrophosphination to produce a phosphine, such as a primary, secondary or tertiary phosphine, for example, useful in catalysis; a bio-α-olefin (and/or bio-based ethylene) can undergo a Diels-Alder reaction to produce a useful ring system; a bio-α-olefin (and/or bio-based ethylene) can undergo a hydroboration to produce an organoborane; a bio-α-olefin (and/or bio-based ethylene) can undergo a hydroacylation reaction to produce a ketone; a bio-α-olefin (and/or bio-based ethylene) can undergo hydration to produce an alcohol; or a bio-α-olefin (and/or bio-based ethylene) can undergo reaction with a hypohalous acid, such as hypochlorous acid, or an equivalent thereof, to produce a halohydrin, many such examples are described herein. Such natural-origin and high modern carbon content bio-molecules produced by these reactions and methods can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Internal olefins can be prepared from bio-α-olefin and/or bio-based ethylene, or blends of such with analogous fossil fuel-derived α-olefins and/or ethylene using, for example, an alkyl halide or triflate and a catalyst, such as palladium in a zero oxidation state. Such a reaction is often referred to as the Heck reaction or the Mizoroki-Heck reaction. Some typical catalysts and precatalysts include tetrakis(triphenylphosphine)palladium(0), palladium chloride, and palladium(II) acetate. Typical supporting ligands are triphenylphosphine, PHOX and BINAP. Typical bases are triethylamine, potassium carbonate, and sodium acetate. The resulting high natural-origin and high modern carbon content internal olefins can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

A halogen, such as Cl, I or Br, and in some cases F, may be added across the double bond of any bio-α-olefin and/or bio-based ethylene, or blends of such with analogous fossil fuel-derived α-olefins and/or ethylene described herein to produce a 1,2-dihalo compound, which are extremely useful in carbon-carbon bond formation. The resulting high natural-origin and modern carbon content bio-based 1,2-dihalo compounds can have, for example, a modern carbon content of greater than about 25 pMC, such as greater than about 35, 45, 50, 55 pMC, such as greater than about 60 percent pMC, such as greater than about 65, 70, 75, 80, 85, 95 pMC or more, such as greater than about 96, 97, 98 or more pMC, such as greater than about 99.5 pMC, or about 100 pMC.

Bio-α-olefins and/or bio-based ethylene, or blends of such with analogous fossil fuel-derived α-olefin and/or ethylene can hydroalkenylate imines or aldehydes to produce amines and alcohols, respectively, as shown in FIG. 3. An example of a catalyst system useful for this transformation is a dual catalysis system with Ni(cod)₂/PCy₃ and either TsNH₂ or PhB(OH)₂. Such bio-amines or bio-alcohols can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Secondary alcohols can be produced from any of the bio-α-olefins and/or bio-based ethylene, or blends of such with analogous fossil fuel-derived α-olefins and/or ethylene described herein by oxymercuration followed by reductive demercuration, often referred to as the oxymercuration-reduction reaction. Typically, for example, this reaction is completed by treating the α-olefin with mercury acetate in wet THF or ether, followed by reduction with, for example, sodium borohydride. The resulting secondary alcohols can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Valuable, useful and strained 4-membered ring ethers (oxetanes) can be prepared by the Paterno-Büchi reaction, which is a photochemical [2+2] cycloaddition reaction of any bio-α-olefins and/or bio-based ethylene, or blends of such with analogous fossil fuel-derived α-olefins and/or ethylene described herein and an aldehyde or ketone. The resulting oxetanes can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Another valuable, useful, and extremely strained ring system that can be produced from any bio-α-olefins and/or bio-based ethylene, or blends of such with analogous fossil fuel-derived α-olefins and/or ethylene described herein is the cyclopropane ring system. Such a reaction can be viewed as insertion of a carbine into the double bond of the α-olefin. Such insertion can be completed, for example, using the Simmons-Smith reaction in which the reactive carbenoid is iodomethylzinc iodide, which is typically formed by the reaction between diiodomethane and a zinc-copper couple. Diazomethane is another such example. Cyclopropanes can be generated using a sulphur ylide in the Johnson-Corey-Chaykovsky reaction. Such bio-cyclopropanes can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Alkylphosphines can be produced from any bio-α-olefins and/or bio-based ethylene, or blends of such with analogous fossil fuel-derived α-olefins and/or ethylene described herein, as shown in FIG. 3. Hydrophosphination or the addition of P-H across the double bond of the α-olefin or ethylene can be completed by using radical initiation processes, such as by using UV light together with the phosphine and the α-olefin, or by using early transition metal catalysts, or a late transition metal catalyst in the presence of the phosphine and the bio-α-olefin. The resulting bio-based phosphines can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Cyclic adducts, Diels-Alder adducts, can be produced using any bio-α-olefins and/or bio-based ethylene, or blends of such with analogous fossil fuel-derived α-olefins and/or ethylene described herein (in this case the dienophile) and a diene, such as 1,3-butadiene or a substituted 1,3-butadiene. Such bio-based Diels-Alder adducts, can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Again, referring to FIG. 3, any bio-α-olefins and/or bio-based ethylene, or blends of such with analogous fossil fuel-derived α-olefins and/or ethylene described herein can be hydroborated, for example, using borane, or an equivalent thereof. In this reaction, the B—H bond is added across the double bond of α-olefin to produce an organoborane. Typically the borane adds in an anti-Markovnikov manner, meaning upon hydrolysis, typically the primary alcohol is produced. The resulting bio-organoboranes can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Ketones can be produced from any bio-α-olefins and/or bio-based ethylene, or blends of such with analogous fossil fuel-derived α-olefins and/or ethylene described herein by adding C—H across the double bond of the selected α-olefin, commonly referred to as hydroacylation. Such reactions are commonly completed utilizing a metal catalyst, such as a rhodium catalyst, such as Wilkinson's catalyst. Such bio-based ketones can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Any bio-α-olefins and/or bio-based ethylene, or blends of such with analogous fossil fuel-derived α-olefins and/or ethylene described herein can be hydrated (addition of water across the double bond). Water can add in a Markovnikov fashion (most stable cation being formed) or in an anti-Markovnikov fashion, thus allowing for the production of primary or secondary alcohols, as shown in FIG. 3. Hydration in a Markovnikov fashion can be completed, for example, by treatment of the α-olefin with aqueous sulfuric acid, while anti-Markovnikov addition can be completed by numerous metal catalyst systems, such as those described in Temkin, O. N., Kinetics and Catalysis, Vol. 55, no. 2 (2014), pp. 172-211. The resulting bio-based alcohols can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

Finally, referring to FIG. 3, halohydrins can be produced from any bio-α-olefins and/or bio-based ethylene, or blends of such with analogous fossil fuel-derived α-olefins and/or ethylene described herein, for example, chlorohydrins, bromohydrins, fluorohydrins or iodohydrins. Halohydrins usually prepared by treatment of desired α-olefin with a halogen, in the presence of water. As described herein, trichloroisocyanuric acid (common swimming pool bleach) is a very useful material to produce chlorohydrins, especially in aqueous acetone solution. The isocyanuric acid that is produced during the reaction is easily removed due to its low solubility. Such high modern carbon content halohydrins can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

The reactions schematically depicted in FIG. 3 may also utilize bio-based ethylene as a starting material. For example, and without limitation, bio-based ethylene may be reacted with a hypohalous acid to form a halohydrin. In some embodiments, bio-based ethylene may be reacted with hypochlorous acid to produce a chloro-ethanol, such as 2-chloroethanol. The resulting bio-based chloro-ethanol can then be further reacted, for example, with a phenoxide ion source to obtain bio-based phenoxyethanol, as described herein.

### Catalyst Two

Oligomerization of the bio-based ethylene of the invention is depicted, for example, in FIG. 1. Catalyst Two (CAT 2) is used to oligomerize the bio-based ethylene to higher α-olefins having four, six, eight, ten, or more carbons. FIG. 1 depicts the oligomerization of bio-based ethylene to the α-olefin, 1-octene. The 1-octene is subsequently oxidized and hydrolyzed to form 1,2-octanediol (caprylyl glycol), a valuable ingredient in, for example, the cosmetics industry. The higher α-olefins produced from bio-based ethylene will also be bio-based compounds. In its limit, the oligomerization proceeds to high molecular weight polyethylene, which is often a competing reaction in the oligomerization reaction.

The oligomerization of ethylene to 1-octene was reviewed by G. P. Belov in Petroleum Chem., Vol. 52, no. 3 (2012), pp. 139-54, the content of which is hereby incorporated by reference in its entirety. The Belov review describes various catalysts, and catalytic methods that may be used to effect the conversion of ethylene to 1-octene. One such catalyst is Chromium(III) acetylacetonate, Cr(acac)₃, a typical octahedral complex containing three acac-ligands. Like most such compounds, it is highly soluble in nonpolar organic solvents. See pages 140-141 of the Belov review.

In certain embodiments, the oligomerization catalyst is in a +3 oxidation state, such as Cr in +3 oxidation state, Mo in a +3 oxidation state or W in a +3 oxidation state, for example, chromium(III) acetylacetonate, Cr(acac)₃, chromium(III) nitrate, chromium(III) acetate, chromium(III) oxide and chromium(III) chloride. In embodiments, the resulting one or more bio-α-olefins have a percent modern carbon (pMC) content that is at least about 20 pMC, such as greater than about 25, 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

A bio-α-olefin may be produced by, for example, introducing PNP ligand and Cr (acac)₃ (0.033 mmol) in a 1:1 ratio in toluene in to a pressure reactor containing a mixture of toluene and methyl aluminoxane (MMAO-12) (1 equivalent of Cr to 300 equivalent of MMAO-12). The pressure reactor may be charged with ethylene, after which the reactor temperature may be maintained between 0-20° C. while the ethylene pressure may be maintained at 1-20 bar. The reaction may be terminated after 1 to 6 hours.

FIGS. 2 and 3 further highlight the many chemical conversions that may be effected using the bio-α-olefins and bio-based ethylene of the invention, as have been described.

### Production of Bio-Based Vinyl Acetate and Bio-Based Ethylene-Vinyl Acetate Copolymers

FIG. 4 schematically depicts another series of useful reactions that utilize the bio-based ethanol and bio-based ethylene, or mixtures of bio-based ethylene and fossil fuel-based ethylene to provide a desired pMC, of the subject invention. In particular, FIG. 4 schematically depicts a method to produce bio-based vinyl acetate monomers and bio-based ethylene-vinyl acetate (EVA) copolymers, for example, from bio-based ethanol.

Vinyl acetate monomers are important and valuable precursors to a myriad of polymeric materials such as, for example, ethylene-vinyl acetate (EVA) copolymers of varying compositions. These EVA copolymers may be blown into a foam utilizing conventional methods and foaming and/or blowing agents, such as carbon dioxide, butane, or azodicarbonamide. EVA copolymers and compositions comprising EVA copolymers and/or EVA copolymer foams may be utilized to manufacture a vast number of consumer goods, such as, for example and without limitation, the soles, midsoles, uppers, and/or bodies of sandals, boots, galoshes, loafers, slippers, moccasins, or athletic, running, leisure, walking, tennis, derby, oxford, slip-on, dress, or casual shoes. Bio-based vinyl acetate, bio-based EVA copolymers, and bio-based EVA copolymer foams may be utilized in industrial and manufacturing processes in the same manner as vinyl acetate, EVA copolymers, and EVA copolymer foams obtained from ancient carbon sources such as fossil fuels. Producing bio-based vinyl acetate, bio-based EVA copolymers, and bio-based EVA foams from organic compounds obtained from renewable sources, e.g., from ethanol obtained from biomass, has the potential to reduce the consumption of fossil fuels, lower the GWP of these syntheses as compared to conventional processes, and to provide for a more sustainable future.

Referring to FIG. 4, bio-based ethanol that was obtained from, for example, biomass material that is non-food biomass (e.g., agricultural or municipal waste) may be dehydrated to make bio-based ethylene utilizing a catalyst (CAT 1) in accordance with the methods that were disclosed and described in detail above and elsewhere herein.

Still referring to FIG. 4, and as described in further detail above and herein, in certain embodiments, CAT 1 may comprise a metal oxide, a silico-aluminate, a silico-aluminophosphate, or a heteropoly acid.

As described above, in some embodiments CAT 1 comprises Al₂O₃, TiO₂—Al₂O₃, SiO₂, SiO₂—Al₂O₃, ZrO₂, WO₃, ZnO/Al₂O₃, MgO—Al₂O₃/SiO₂, USY, or ZSM5. In embodiments where the catalyst comprises ZSM5, the ZSM5 may have a Si/Al ratio that is 20:1 to 360:1 (such ratios are typically denoted as “20” or “360”); in other embodiments, the ZSM5 has a Si/Al ratio of 19:1 (or 19).

As described above, in further embodiments CAT 1 is a zeolite treated with H₃PO₄.

As described above, methods of the present invention also include embodiments where CAT 1 comprises molybdophosphoric acid or tungstophosphoric acid.

Still referring to FIG. 4, bio-based vinyl acetate may be made from the bio-based ethylene produced from the catalytic dehydration of bio-based ethanol. In certain embodiments, the synthesis of bio-based vinyl acetate may be effected through the reaction of bio-based ethylene with acetic acid and oxygen in the presence of a catalyst (CAT 3).

In further embodiments, the acetic acid used to synthesize bio-based vinyl acetate may be bio-acetic acid that is obtained through the hydrocarboxylation of bio-based ethylene using hydrogen, carbon dioxide and a catalyst. For example, a suitable catalyst for the hydrocarboxylation of bio-based ethylene is a combination of [RhCl(cod)]2 (cod=cyclooctadiene) as a catalyst and diethylzinc as a hydride source. However, this is not required, and acetic acid from any source may be utilized to synthesize bio-based vinyl acetate from bio-based ethylene.

### Catalyst Three

An industrial-scale process for the synthesis of vinyl acetate using gaseous ethylene, acetic acid, oxygen, and palladium-based catalysts is discussed by A. C. Dimian and C. S. Bildea in Chemical Process Design: Computer-Aided Case Studies, ISBN: 978-3-527-31403-4, pp. 287-311, Wiley-VCH (Weinheim, Germany, 2008), the content of which is incorporated herein in its entirety.

A vinyl acetate synthesis process utilizing the non-catalytic cracking of soybean oil to produce acetic acid is discussed by B. Jones et al. in The Production of Vinyl Acetate Monomer as a Co-Product from the Non-Catalytic Cracking of Soybean Oil, Processes, Vol. 3, no. 3 (August 2015), pp. 619-33, the content of which is incorporated herein in its entirety. The process described by Jones et al. also utilized ethylene, acetic acid, and oxygen as reagents with a palladium-based catalyst.

Still referring to FIG. 4, in certain embodiments CAT 3 is a palladium-based catalyst. As described by Dimian and Bildea, suitable palladium-based catalysts may be impregnated on silica and metal acetates may be employed as activators, such as, for example and without limitation, potassium acetate. In other embodiments, noble metals (e.g., Au) may be employed as enhancers. As further described by Dimian and Bildea, a typical “Bayer-type” catalyst may consist of 0.15-1.5 weight percent palladium, 0.2-1.5 weight percent gold, and 4-10 weight percent potassium acetate on spherical silica particles of 5 mm diameter. Palladium chloride, palladium acetate, and copper chloride may also be suitable catalysts for the synthesis of bio-based vinyl acetate.

The synthesis of vinyl acetate is typically carried out in the gas phase in order to improve yield and mitigate catalyst corrosion issues. Typical reactors include plug-flow reactors (PFR) or fluidized bed reactors. As disclosed by Dimian and Bildea, preferred reaction conditions are temperatures around 150-160° C. with pressures of 8-10 bar. The ethylene to acetic acid ratio in the reactor should be about 2:1 to about 3:1, with a preferable ethylene/acetic acid ratio of about 3:1. Oxygen concentration should be kept below 8% by volume based on an acetic-acid-free mixture in order to mitigate explosion risks and to minimize the undesirable oxidation of ethylene as a side reaction. The low concentration of oxygen limits the single-pass yield of the reaction, typically necessitating large recycle loops to achieve the desired yield of vinyl acetate.

Dimian and Bildea disclose using a spherical palladium-based catalysts of 5 mm diameter in a PFR with a bed void fraction of 45%, and further disclose introducing the gaseous reagents with an inlet pressure of 10 bar at a velocity of 0.5 m/s. The resultant outlet stream is flash separated at 33° C. and 9 bar and further separation and recycle steps are performed in order to obtain the vinyl acetate at an acceptable purity.

Such a high modern carbon content, natural-origin bio-based vinyl acetate can have, for example, a modern carbon content of greater than about 25 pMC, such as greater than about 35, 45, 50, 55 pMC, such as greater than about 60 pMC, such as greater than about 65, 70, 75, 80, 85, 95 pMC or more, such as greater than about 96, 97, 98 or more pMC, such as greater than about 99.5 pMC, or about 100 pMC.

### Catalyst Four

As further depicted in FIG. 4, bio-based vinyl acetate may be further reacted with bio-based ethylene and a catalyst (CAT 4) to produce a bio-based ethylene-vinyl acetate (EVA) copolymer.

The bio-based ethylene utilized for this copolymerization may be obtained in the manner described in detail above, namely, the catalytic dehydration of bio-based ethanol that was obtained from a renewable source, such as, for example, non-food biomass or lignocellulosic material. Suitable catalysts for this dehydration include all of the catalysts disclosed above as CAT 1.

Methods and processes for polymerizing ethylene and vinyl acetate are described in U.S. Pat. No. 2,703,794, the disclosure of which is incorporated by reference herein in its entirety.

T. Saegusa et al. described the use of a triethylaluminum, zinc chloride, and carbon tetrachloride (AlEt₃-ZnCl₂—CCl₄) catalyst to polymerize ethylene and vinyl acetate in Alternating Copolymerization of Ethylene with Vinyl Acetate by AlEt₃-ZnCl₂—CCl₄ Catalyst, Polymer J., Vol. 8 (1976), pp. 593-600, the content of which is incorporated herein in its entirety.

Still referring to FIG. 4, in certain embodiments CAT 4 is a composite reduction-oxidation (“redox”) catalyst comprising, for example, a peroxygen compound. M. Roedel disclosed the following as suitable peroxide compounds for this purpose: salts of hydrogen peroxides, perborates, percarbonates, persulfates, perphosphates, percarboxylates; organic hydroperoxides such as methyl hydroperoxide, ethyl hydroperoxide, tertiary butyl hydroperoxide, tetralin hydroperoxide, cumene hydroperoxide, cyclohexane hydroperoxide, cyclohexanone peroxide; diacyl peroxides such as benzoyl peroxide, acetyl peroxide, acetyl benzoyl peroxide, lauroyl peroxide, crotonyl peroxide, etc.; alkyl acyl peroxides such as tertiary butyl perbenzoate, ditertiary butyl perphthalate, tertiary butyl permaleic acid, perbenzoic acid, diisobutylene ozonide, methyl ethyl ketone peroxide, acetone methyl isobutyl ketone peroxide, succinic acid peroxide, methyl isobutyl ketone peroxide, polyperoxides, diethyl peroxydicarbonate, pelargonyl peroxide, and the like.

As described in U.S. Pat. No. 2,703,794, CAT 4 may further comprise one or more of a metal salt, heavy metal ions which may exist in more than one valence state, and an organic reducing agent. Roedel further discloses in U.S. Pat. No. 2,703,794 that salts of Group I-B and VIII metals are well-suited for this purpose, and appropriate organic compounds include sulfinic acids, benzoin, 1-ascorbic acid, primary, secondary, tertiary and polyamines, sodium or zinc formaldehyde sulfoxylate and alkanolamines, such as triethanolamine.

In other embodiments, CAT 4 may comprise triethylaluminum, zinc chloride, and carbon tetrachloride (AlEt₃-ZnCl₂—CCl₄), as described by T. Saegusa et al.

In some embodiments, the mole percentage of bio-based ethylene in the bio-based EVA copolymer produced by the copolymerization process described above may be in the range of about 70 mole percent to about 98 mole percent.

In some embodiments, the mole percentage of bio-based vinyl acetate in the bio-based EVA copolymer produced by the copolymerization process described above may be in the range of about 2 mole percent to about 30 mole percent.

In certain embodiments, the bio-based ethylene-vinyl acetate (EVA) copolymer, has at least about 20 pMC, at least about 30 pMC, at least about 40 pMC, at least about 50 pMC, at least about 60 pMC, at least about 70 pMC, at least about 80 pMC, at least about 90 pMC, or at least about 100 pMC. In particular embodiments, the pMC is at least about 50 pMC and in other particular embodiments the pMC is nearly or about 100 pMC.

In certain embodiments, the mole percentage of bio-ethylene monomer in the composition is in the range of about 70% to about 98%, such as between about 75 and about 95 percent, between about 78 and about 94 percent or between about 80 and about 92 percent. In certain embodiments, the mole percentage of vinyl acetate monomer in the composition is in the range of about 2% to about 30%, such as between about 3 and about 27 percent, between about 4 and about 25 percent or between about 7 and about 20 percent.

In some embodiments, the composition comprising the bio-based EVA copolymer may be further processed in to a bio-based EVA copolymer foam utilizing conventional methods and blowing and/or foaming agents, such as carbon dioxide, butane, or azodicarbonamide.

In some embodiments, the density of the bio-based EVA foam is between about 0.2 and about 0.8 g/cm³, such as between about 0.3 and about 0.7 g/cm³ or between about 0.35 and about 0.6 g/cm³.

In other embodiments, the bio-based polyvinyl acetate homopolymers or bio-based EVA copolymers may be hydrolyzed to form bio-based polyvinyl alcohol or bio-based ethylene vinyl alcohol compounds with a high percent modern carbon (pMC).

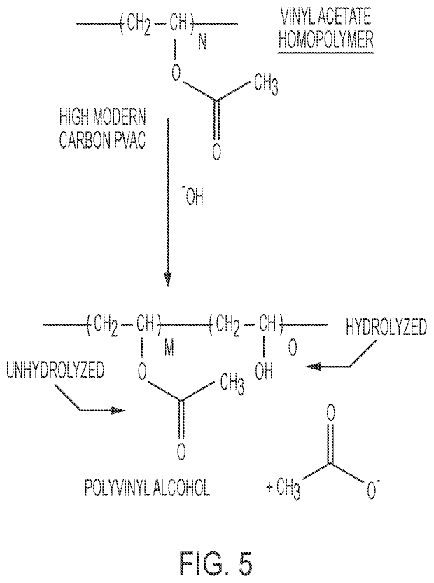

For example, FIG. 5 schematically depicts how bio-based polyvinyl acetate homopolymer can be hydrolyzed, for example, using sodium or potassium hydroxide in aqueous or alcoholic solutions, to produce a bio-based vinyl acetate/vinyl alcohol copolymer, e.g., polyvinyl alcohol, and acetate monomer with high pMC. As depicted in FIG. 5, the bio-based vinyl acetate homopolymer may be composed of N repeating vinyl acetate monomers. Following hydrolysis, the resulting bio-based polyvinyl alcohol compound may be composed of M mole-percent of “unhydrolyzed” vinyl acetate monomers and O mole-percent “hydrolyzed” ethanol monomers. Various levels of hydrolysis of the bio-based polyvinyl acetate homopolymer are possible from 100 percent (or nearly so) to 1 percent or less, for example, greater than 0.5, 1, 2, 3, 4, 5, 10, 20, 40, 60, 80, 90, or greater than 99 percent hydrolyzed. That is, 0 may be any value in the range of about 0 mole percent to about 100 mole percent.

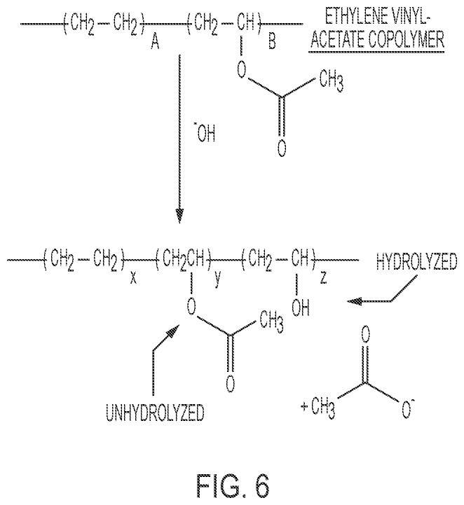

By way of further example, and referring now to FIG. 6, a bio-based vinyl acetate copolymer, such as bio-based EVA (as shown in FIG. 6) can likewise be hydrolyzed to form a bio-based ethylene vinyl alcohol copolymer with a high pMC. The bio-based EVA copolymer may be composed of A mole-percent of ethylene monomers and B mole-percent of vinyl acetate monomers. The hydrolysis of the EVA copolymer may be accomplished by, for example, using sodium or potassium hydroxide in aqueous or alcoholic solutions. Following hydrolysis, the resulting bio-based ethylene vinyl alcohol copolymer may be composed of X mole-percent of ethylene monomers, Y mole-percent of “unhydrolyzed” vinyl acetate monomers, and Z mole-percent of “hydrolyzed” ethanol monomers. Various levels of hydrolysis of the bio-based EVA copolymer are possible from 100 percent (or nearly so) to 1 percent or less, for example, greater than 0.5, 1, 2, 3, 4, 5, 10, 20, 40, 60, 80, 90, or greater than 99 percent hydrolyzed. That is, Z may be any value in the range of about 0 mole percent to about 100 mole percent, where X+Y+Z=100 mole percent.

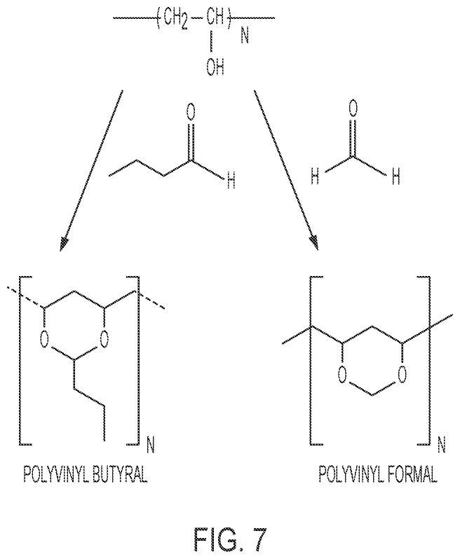

FIG. 7 schematically depicts example reactions in which bio-based polyvinyl alcohol polymers may be further functionalized. For example, bio-based polyvinyl alcohol with N repeating monomeric units may be reacted with butyraldehyde to produce bio-based polyvinyl butyral with a high pMC and N repeating monomeric units. As an additional non-limiting example, bio-based polyvinyl alcohol with N repeating monomeric units may be reacted with formaldehyde to produce polyvinyl formal with N repeating monomeric units.

### Biomass Sources

Ethanol produced from any biomass materials or biomass-derived materials can be used to make bio-based ethylene and bio-based polymers (e.g., bio-based polyethylene) according to the processes described herein.

A typical biomass resource contains cellulose, hemicellulose, and lignin, plus lesser amounts of proteins, extractables, and minerals. The complex carbohydrates contained in the cellulose and hemicellulose fractions can be converted into sugars, e.g., fermentable sugars, by saccharification, and the sugars can then be converted by further processing, e.g., fermentation or hydrogenation, into a variety of products, such as alcohols or organic acids. For example, in lignocellulosic ethanol production, cellulases secreted by cellulolytic microorganisms convert cellulosic materials to glucose, and then glucose is fermented into ethanol.

As used herein, the terms biomass and biomass materials include lignocellulosic, cellulosic, starchy, and microbial materials. Lignocellulosic materials comprise different combinations of cellulose, hemicellulose, and lignin.

Lignocellulosic materials include, but are not limited to, wood, particle board, forestry wastes (e.g., sawdust, aspen wood, wood chips), grasses, (e.g., switchgrass, miscanthus, cord grass, reed canary grass), grain residues, (e.g., rice hulls, oat hulls, wheat chaff, barley hulls), agricultural waste (e.g., silage, canola straw, wheat straw, barley straw, oat straw, rice straw, jute, hemp, flax, bamboo, sisal, abaca, corn cobs, corn stover, soybean stover, corn fiber, alfalfa, hay, coconut hair), sugar processing residues (e.g., bagasse, beet pulp, agave bagasse), algae, seaweed, manure, sewage, and mixtures of any of these. In further embodiments, the biomass can be lignin hulls. Lignin hulls are the material that is remaining after a lignocellulosic biomass has been saccharified.

In some embodiments, the lignocellulosic material includes corn cobs. Ground or hammermilled corn cobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant. Advantageously, no additional nutrients (other than a nitrogen source, e.g., urea or ammonia) are required during fermentation of corn cobs or cellulosic or lignocellulosic materials containing significant amounts of corn cobs.

Cellulosic materials include, for example, paper, paper products, paper waste, paper pulp, pigmented papers, loaded papers, coated papers, filled papers, magazines, printed matter (e.g., books, catalogs, manuals, labels, calendars, greeting cards, brochures, prospectuses, newsprint), printer paper, polycoated paper, card stock, cardboard, paperboard, materials having a high alpha-cellulose content such as cotton, and mixtures of any of these. Further examples of paper products are described in U.S. Patent App. Pub. No. 2013-0052687, filed Feb. 14, 2012, the disclosure of which is incorporated herein by reference. Cellulosic materials can also include lignocellulosic materials which have been de-lignified.

Starchy materials include starch itself (e.g., corn starch, wheat starch, potato starch and rice starch), a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils, and/or peas. Blends of any two or more starchy materials are also starchy materials. Mixtures of starchy, cellulosic, and/or lignocellulosic materials can also be used. For example, a biomass can be an entire plant, a part of a plant, or different parts of a plant, e.g., a wheat plant, cotton plant, a corn plant, rice plant, or a tree.

Microbial materials include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture and fermentation systems.

The diversity of biomass materials may be further increased by pretreatment, for example, by changing the crystallinity and/or molecular weights of the organic polymers within the biomass.

In other embodiments, the biomass materials, such as cellulosic, starchy, and lignocellulosic feedstock materials, can be obtained from transgenic microorganisms and plants that have been modified with respect to a wild-type variety. Such modifications may be, for example, through the iterative steps of selection and breeding to obtain desired traits in a plant. Furthermore, the plants may have had genetic material removed, modified, silenced and/or added with respect to the wild-type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogenous genes. The artificial genes can be created by a variety of ways including treating the plant or seeds with, for example, chemical mutagens (e.g., using alkylating agents, epoxides, alkaloids, peroxides, formaldehyde), irradiation (e.g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers. Additional genetically modified materials have been described in U.S. Patent Appl. Publ. No. 2013-0052687, filed Feb. 14, 2012, incorporated by reference above.

Ethanol can be produced from mixtures of any biomass materials, including mixtures of any of the biomass materials described herein.

### Biomass Treatment and Preparation

The biomass may undergo mechanical treatments, such as milling, grinding, cutting, pressing, shearing, or chopping, and/or electron bombardment, as described in U.S. Application Publication Nos. 2012/0100577 and 2014/0011258, and U.S. Pat. Nos. 7,932,065, 9,644,244, 9,677,039, 9,677,039, and 9,816,231, the entire disclosures of which are incorporated herein by reference.

### Saccharification

In order to convert a feedstock to a form that can be readily processed, the glucan- or xylan-containing cellulose in the feedstock can be hydrolyzed to low molecular weight carbohydrates, such as sugars, by a saccharifying agent, e.g., an enzyme or acid, a process referred to as saccharification. For example, the feedstock can be hydrolyzed using one or more enzymes, e.g., by combining the feedstock materials and the enzyme(s) in a solvent or fluid medium, e.g., in an aqueous solution. The low molecular weight carbohydrates can then be used to make ethanol. In some cases, the feedstock is boiled, steeped, or cooked in hot water prior to saccharification, as described in U.S. Patent Application Publication No. 2012/0100577, the entire content of which is incorporated herein.

Specifically, the enzymes can be supplied by organisms that are capable of breaking down biomass (such as the cellulose and/or the lignin portions of the biomass), or that contain or manufacture various cellulolytic enzymes (cellulases), ligninases, or various small molecule biomass-degrading metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (betaglucosidases).

During saccharification, a cellulosic substrate can be initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally, cellobiase cleaves cellobiose to yield glucose. The efficiency (e.g., time to hydrolyze and/or completeness of hydrolysis) of this process depends on the recalcitrance of the cellulosic material.

The saccharification process can be partially or completely performed in a tank (e.g., a tank having a volume of at least 4000, 40,000, or 500,000 L) in a manufacturing plant, and/or can be partially or completely performed in transit, e.g., in a rail car, tanker truck, or in a supertanker or the hold of a ship. If saccharification is performed in a manufacturing plant under controlled conditions, the cellulose may be substantially entirely converted to sugar, e.g., glucose, in about 12-96 hours. If saccharification is performed partially or completely in transit, saccharification may take longer.

It is generally preferred that the tank contents be mixed during saccharification, e.g., using jet mixing as described in International Application Publication. No. WO/2010/135380, the full disclosure of which is incorporated by reference herein.

The addition of surfactants can enhance the rate of saccharification. Examples of surfactants include non-ionic surfactants, such as a Tween® 20 or Tween® 80 polyethylene glycol surfactants, ionic surfactants, or amphoteric surfactants.

In some embodiments, it is generally preferred that the concentration of the sugar solution resulting from saccharification be relatively high, e.g., greater than 40%, or greater than 50, 60, 70, 80, 90 or even greater than 95% by weight. Water may be removed, e.g., by evaporation, to increase the concentration of the sugar solution. Water removal reduces the volume, and also inhibits microbial growth in the solution.

Alternatively, sugar solutions of lower concentrations may be used, in which case it may be desirable to add an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during transport and storage, and can be used at appropriate concentrations, e.g., between 15 and 1000 ppm by weight, e.g., between 25 and 500 ppm, or between 50 and 150 ppm. If desired, an antibiotic can be included even if the sugar concentration is relatively high. Alternatively, other additives with anti-microbial of preservative properties may be used. The antimicrobial additive(s) may be food-grade.

A relatively high concentration solution can be obtained by limiting the amount of water added to the biomass material with the enzyme. The concentration can be controlled, e.g., by controlling how much saccharification takes place. For example, concentration can be increased by adding more biomass material to the solution. In order to keep the sugar that is being produced in solution, a surfactant can be added, e.g., one of those discussed above. Solubility can also be increased by increasing the temperature of the solution. For example, the solution can be maintained at a temperature of 40-50° C., 60-80° C., or even higher.

Suitable cellulolytic enzymes to use as saccharifying agents include cellulases from species in the genera Bacillus, Coprinus, Myceliophthora, Cephalosporium, Scytalidium, Penicillium, Aspergillus, Pseudomonas, Humicola, Fusarium, Thielavia, Acremonium, Chlysosporium and Trichoderma, especially those produced by a strain selected from the species Aspergillus (see, e.g., EP Pub. No. 0 458 162), Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium spp. (including, but not limited to, A. persicinum, A. acremonium, A. brachypenium, A. dichromosporum, A. obclavatum, A. pinkertoniae, A. roseogriseum, A. incoloratum, and A. furatum). Preferred strains include Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H.

Cellulolytic enzymes may also be obtained from Chrysosporium, preferably a strain of Chrysosporium lucknowense. Additional strains that can be used include, but are not limited to, Trichoderma (particularly T. viride, T. reesei, and T. koningii), alkalophilic Bacillus (see, e.g., U.S. Pat. No. 3,844,890 and EP Pub. No. 0 458 162), and Streptomyces (see, e.g., EP Pub. No. 0 458 162).

### Fermentation of Biomass to Produce Bio-Based Ethanol

Biomass or saccharified biomass can be fermented to produce bio-based ethanol. Alternatively, after saccharification or other processing of the biomass, sugars (e.g., sucrose, glucose, and/or xylose) can be purified or isolated, and the purified or isolated sugars can be fermented to produce bio-based ethanol. For example, sugars can be purified or isolated by precipitation, crystallization, chromatography (e.g., simulated moving bed chromatography, high pressure chromatography), centrifugation, extraction, any other purification or isolation method known in the art, and combinations thereof. Isolating or purifying sugars before fermentation may increase the purity of the bio-based ethanol produced.

Yeast and Zymomonas bacteria, for example, can be used for fermentation to convert sugar(s) to alcohol(s). Other microorganisms are discussed below. The optimum pH for fermentations is about pH 4 to 7. For example, the optimum pH for yeast is from about pH 4 to 5, while the optimum pH for Zymomonas is from about pH 5 to 6. Typical fermentation times are about 24 to 168 hours (e.g., 24 to 96 hours) with temperatures in the range of 20° C. to 40° C. (e.g., 26° C. to 40° C.), however thermophilic microorganisms prefer higher temperatures.

In some embodiments, e.g., when anaerobic organisms are used, at least a portion of the fermentation is conducted in the absence of oxygen, e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic condition, can be achieved or maintained by carbon dioxide production during the fermentation and no additional inert gas is needed.

Jet mixing may be used during fermentation, and in some cases saccharification and fermentation are performed in the same tank.

Nutrients for the microorganisms may be added during saccharification and/or fermentation, for example the food-based nutrient packages described in U.S. Patent Appl. Pub. No. 2012/0052536, filed Jul. 15, 2011, the complete disclosure of which is incorporated herein by reference.

Fermentation includes the methods and products that are disclosed in International Application Publication Nos. WO/2013/096700, WO/2013/096703, and WO/2013/096693, the contents of which are incorporated by reference herein in their entireties.

Mobile fermenters can be utilized, as described in U.S. Pat. No. 8,318,453, the disclosure of which is incorporated herein in its entirety. Similarly, the saccharification equipment can be mobile. Further, saccharification and/or fermentation may be performed in part or entirely during transit.

The microorganism(s) used in fermentation can be naturally-occurring microorganisms and/or engineered microorganisms. For example, the microorganism can be a bacterium (including, but not limited to, e.g., a cellulolytic bacterium), a fungus, (including, but not limited to, e.g., a yeast), a plant, a protist, e.g., a protozoa or a fungus-like protist (including, but not limited to, e.g., a slime mold), or an algae. When the organisms are compatible, mixtures of organisms can be utilized.

Suitable fermenting microorganisms have the ability to convert carbohydrates, such as glucose, fructose, xylose, arabinose, mannose, galactose, oligosaccharides and/or polysaccharides into fermentation products, including ethanol. Fermenting microorganisms include strains of the genus Saccharomyces spp. (including, but not limited to, S. cerevisiae (baker's yeast), S. distaticus, S. uvarum), the genus Kluyveromyces, (including, but not limited to, K. marxianus, K fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii (Philippidis, G. P., 1996, Cellulose bioconversion technology, in Handbook on Bioethanol: Production and Utilization, Wyman, C. E., ed., Taylor & Francis, Washington, D.C., 179-212)). Other suitable microorganisms include, for example, Zymomonas mobilis, Clostridium spp. (including, but not limited to, C. thermocellum (Philippidis, 1996, supra), C. saccharobutylacetonicum, C. saccharobutylicum, C. Puniceum, C. beijernckii, and C. acetobutylicum), Moniliella pollinis, Moniliella megachiliensis, Lactobacillus spp. Yarrowia lipolytica, Aureobasidium sp., Trichosporonoides sp., Trigonopsis variabilis, Trichosporon sp., Moniliellaacetoabutans sp., Typhula variabilis, Candida magnoliae, Ustilaginomycetes sp., Pseudozyma tsukubaensis, yeast species of genera Zygosaccharomyces, Debaryomyces, Hansenula and Pichia, and fungi of the dematioid genus Torula. For instance, Clostridium spp. can be used to produce ethanol. Preferred microorganisms include Saccharomyces spp., especially strains genetically modified to ferment all sugars, including xylose and arabinose.

Many such microbial strains are publicly available, either commercially or through depositories such as the ATCC (American Type Culture Collection, Manassas, Va., USA), the NRRL (Agricultural Research Service Culture Collection, Peoria, Ill., USA), or the DSMZ (Deutsche Sammlung von Mikroorganismen and Zellkulturen GmbH, Braunschweig, Germany), to name a few.

Commercially available yeasts include, for example, Red Star®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

In addition, suitable xylose and glucose fermenting strains are commercially available from Royal DSM, Lallemand, and LEAF.

Many microorganisms that can be used to saccharify biomass material and produce sugars can also be used to ferment and convert those sugars to bio-based ethanol.

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

Also following fermentation, the residual solid biomass products may be combusted in order to provide heat to generate power, for example in a combined heat and power generator system, for the processing steps heretofore described, as well as additional processing steps to convert the bio-based ethanol to further bio-based carbon compounds with a high pMC content. Burning the residual solid biomass products increases the energy efficiency of the process and could potentially reduce a production facility's reliance on electricity from an electrical grid. This may also the consumption of fossil fuels and other non-renewable sources of carbon (e.g., coal), as electricity sourced from the grid may be produced through the combustion of these non-renewable fuel sources.

### EXAMPLES

The following examples serve only to illustrate the invention and practice thereof. The examples are not to be construed as limitations on the scope or spirit of the invention.

### Example 1

Catalyst One Preparation: 1% La—γAl₂O₃ A calculated amount of Lanthanum (III) Nitrate (Strem Chemicals) was dissolved in 100 mL of deionized water; the calculated amount correlated to the amount providing 1% La in the final product. For example, for 100 grams of catalyst, an amount of Lanthanum (III) Nitrate providing 1 gram La (1% of 100 grams) would be used. γAl₂O₃ (Strem Chemicals) was added to the salt solution and the solution was incubated on a shaker at 50° C. for 24 hours. Following incubation, the solution was decanted from the catalyst and the catalyst was rinsed twice with 100 mL of deionized water. The final catalyst was dried at 110° C. overnight under vacuum.

### Example 2

Catalyst One Preparation: 1% La—ZSM5 A calculated amount of Lanthanum (III) Nitrate (Strem Chemicals) was dissolved in 100 mL of deionized water, as described above. ZSM5 (ACS Material®) was added to the salt solution and the solution was incubated on a shaker at 50° C. for 24 hours. Following incubation, the solution was decanted from the catalyst and the catalyst was rinsed twice with 100 mL of deionized water. The final catalyst was dried at 110° C. overnight under vacuum. ZSM5 (Si/A1-19) and ZSM5 (Si/A1-360) were used in separate preparations.

### Example 3

Catalyst One Preparation: 1% La—USY—Al₂O₃ A calculated amount of Lanthanum (III) Nitrate (Strem Chemicals) was dissolved in 100 mL of deionized water, as described above. USY—Al₂O₃ (80% USY—20% Al₂O₃, Grace Davison) was added to the salt solution and the solution was incubated on a shaker at 50° C. for 24 hours. Following incubation, the solution was decanted from the catalyst and the catalyst was rinsed twice with 100 mL of deionized water. The final catalyst was dried at 110° C. overnight under vacuum.

### Example 4

Bio-based Ethanol to Bio-based Ethylene Reaction: Various catalysts, including each of the catalysts from Examples 1-3, were used to dehydrate bio-based ethanol to generate bio-based ethylene. The bio-based ethanol used in these reactions was cellulosic ethanol, produced according to the methods described herein. Typically very pure bio-based ethanol was obtained, for example, ethanol accounting for 99.4% of the volatile components in the resulting mixture, as determined by flame ionization detection. The amount of water present was 8 percent by weight.

Each catalyst reaction was performed at a temperature of 400° C., N₂ gas flow rate of 50 mL/min, and a cellulosic ethanol flow rate of 0.30 mL/min, as follows: The catalyst was added to a fixed bed reactor at a volume of 11 cc. Before each reaction, the catalyst was pre-activated at 400° C., N₂ at 50 mL/min, for 2 hours. Ethanol dehydration was carried out in the fixed bed reactor at a temperature of 400° C. and at ambient pressure using N₂ flow rate of 50 mL/min. Preheated (60° C.) cellulosic ethanol was injected into the catalyst bed with a flow rate of 0.30 mL/min.

The reactor's exit flow was connected to a gas-liquid separator, which maintained a temperature of 5° C. The gas from this separator flowed to another flask at 5° C. to condense trace amount liquids. The final gas was dried before entering the polymerization reactor using Molecular Sieve-3 A.

Table 1 provides the conversion rate and ethylene molar selectivity for the different catalyst reactions.

TABLE 1

| | Ethanol | Ethane | Ethylene | Propane | Butene | Unknown |
|---|---|---|---|---|---|---|
| | Conversion | mole | mole | mole | mole | mole |
| Catalyst One | (%) | (%) | (%) | (%) | (%) | (%) |
| γAl₂O₃ | 99.7 | 0.2 | 62.4 | 0 | 0.4 | 0.50 |
| γAl₂O₃ calcined @ | 99.9 | 0 | 59.1 | 0 | 0 | 0.50 |
| 850° C. | | | | | | |
| 1% La - γAl₂O₃ | 99.9 | 0 | 70.1 | 0 | 0 | 0 |
| (Example 1) | | | | | | |
| ZSM5 | 35.6 | 0.1 | 15.4 | 0 | 1.0 | 5.0 |
| (Si/Al-360) | | | | | | |
| 1% La - ZSM5 | 99.8 | 0.5 | 53.7 | 0 | 8.1 | 13.4 |
| (Si/Al-360) | | | | | | |
| (Example 2) | | | | | | |
| 1% La - ZSM5 | 99.9 | 0.7 | 5.6 | 26.8 | 1.1 | 1.9 |
| (Si/Al-19) | | | | | | |
| (Example 2) | | | | | | |
| 80% USY - 20% | 99.7 | 0.5 | 63.5 | 0 | 0 | 0 |
| Al₂O₃ | | | | | | |
| 1% La - USY - | 99.6 | 0.05 | 66.2 | 0 | 0 | 0 |
| Al₂O₃ | | | | | | |
| (Example 3) | | | | | | |

### Example 5

Ethylene Polymerization to Polyethylene: The bio-based ethylene gas produced using the catalyst of Example 1 was used for the polymerization reaction. In addition, a separate polymerization reaction was performed with bottled ethylene (Praxair), which is ethylene obtained from petroleum.

A 1 L round bottom flask equipped with a gas inlet was charged with 30 mg of Cp₂ZrCl₂ (Sigma Aldrich). 275 mL of dry toluene (Sigma Aldrich) and 25 mL of 2M trimethylaluminum (Sigma Aldrich) were added. The mixture was purged with N₂ for 30 minutes, then the mixture was allowed to pre-react for 20 minutes at 60° C. After saturating the toluene solution with ethylene, 0.7 mL of 7% methyl aluminoxane (MMAO-7, Sigma Aldrich) was injected stepwise. The polymerization activity was followed by monitoring the rate of ethylene uptake. During the reaction, the supply of ethylene gas slightly exceeded demand. When the reaction mixture became thick with product, the rate of ethylene uptake decreased substantially. At this point (after about 24 hours reaction time), the ethylene flow was turned off and N₂ flow was started to purge the reaction.

The reaction mixture was hydrolyzed by the addition of several 2 mL aliquots of ethanol. The flask was then cooled in an ice bath. The reaction mixture was filtered and the solid polymer was washed with ethanol. The solid polymer was placed in 100 mL of ethanol and sonicated for 1 hour at 50° C., then the solid was filtered and washed with ethanol. This final solid was dried at 50° C. overnight under vacuum. For the reaction using the bio-based ethylene from Example 1, 29.6 grams of bio-based polyethylene was obtained.

Radiocarbon testing was performed to confirm that the carbon in the bio-based polyethylene made by polymerizing the bio-based ethylene of Example 1 is bio-based carbon. 12.9 mg of the polyethylene was analyzed using ASTM D6866-18 Method B (AMS). The percent modern carbon (pMC) was determined as the percentage of Carbon-14 measured in the polyethylene sample relative to the Carbon-14 of a modern reference standard (NIST 4990C). The percent bio-based carbon content was calculated from pMC by applying a small adjustment factor for Carbon-14 in carbon dioxide in air today. The results of the testing were: 99.62±0.28 pMC, 100% bio-based carbon content (as a fraction of total organic carbon). In contrast, radiocarbon testing results for the polyethylene made from Praxair ethylene indicated <0.44 pMC and 0% bio-based carbon content (as a fraction of total organic carbon).

### Example 6

Production of 1,2-diols from Corn Cob Biomass: Corn cob was hammer-milled to approximate dimensions of 1×1×1 mm. Hammer-milled corn cob was irradiated using an e-beam operating at 1 MV and a beam current of 50 mA (50 kW) to a dose of about 35 Mrad (350 kGy). Treated corn cob was saccharified using enzymes to produce approximately 100 g/L of total sugars. C5/C6 fermenting yeast was added to the reaction vessel to produce approximately 50 g/L of ethanol. Solids were removed using a filter belt and 2 UF stages and the ethanol was vacuum distilled to yield 95 percent ethanol. The ethanol was dehydrated to yield ethylene gas (utilizing the same catalyst as in the ethylene/polyethylene reaction). Ethylene gas was oligomerized using [Cr(PNP)Cl₂(μ-Cl₂)]₂ at 50 bar and 50° C. to give 55 percent by CAT 2 weight 1-octene and 24 percent by weight 1-hexene, which were purified by distillation. The hexene and octene fractions were dihydroxylated with osmium tetroxide, water and hydrogen peroxide. The 1,2-diols were separated by distillation.

### Example 7

Preparation of N,N-bis(diphenylphosphino)isopropylamine (PNP), a Ligand for Ethylene Oligomerization: Chlorodiphenyl phosphine (75 mL, 89.55 g, 405 mmol, 2 eq) was charged to a 3 necked round bottom flask under a nitrogen atmosphere, along with 250 mL of methylene chloride and 75 mL of triethylamine. The reaction was cooled on an ice bath, and isopropylamine (18 mL, 11.96 g, 203 mmol, 1 eq) was added slowly over the course of 20 minutes via an addition funnel. Once addition was complete, the reaction was removed from the ice bath, and allowed to stir overnight at room temperature. The reaction mixture was filtered through a glass frit to remove triethylamine hydrochloride, and the residue evaporated under reduced pressure. The resultant light yellow solid was recrystallized from 4 volumes of acetonitrile to yield 42.6 g (49.3%) of the title compound as a white solid. (melting point, 135.4° C.; 1H NMR—δ 1.05-1.07 (d) 6H; 3.68 (m) 1H, 7.32-7.48 (m) 20H; 13C NMR δ 24.5 (t) CH₃; 57.8 (t) CH; 139.75 (d) P—C; 132.6 (d) ortho CH; 128.7 (d) meta CH; 129.29 (s) para CH).

### Example 8

Ethylene Polymerization to Polyethylene: Materials used for the polymerization: Cr(acac)₃; modified methyl aluminoxane (MMAO-12, Sigma Aldrich); toluene (Sigma Aldrich); Bis(diphenylphosphino)isopropylamine (PNP) ligand was synthesized in the laboratory and the method of preparation was explained above.

A solution of 125 mg of PNP ligand in 20 mL toluene was added to a solution of 55 mg Cr(acac)₃ in 20 mL toluene in a 100 mL round bottom flask under highly inert atmosphere. The mixture was stirred for 5 minutes at ambient temperature and then transferred to a pressure reactor (autoclave) containing a mixture of 160 mL toluene and 8.25 mL of MMAO-12 at 60° C. Initially the reactor was pressurized to 40 psi with hydrogen, then switched to ethylene and reactor pressure was maintained at 600 psi. During the reaction the temperature was increased 105° C. The reaction was terminated after 120 minutes by discontinuing the ethylene feed to the reactor and cooling the reactor to below 10° C. After cooling down, the reactor was depressurized and the polymer was collected. The polymer was washed with ethanol followed by 10% aqueous hydrochloric acid and water. The solid was filtered and the products were dried overnight in an oven at 50° C. under vacuum. The weight of the recovered polymer was 245 grams.

### Example 9

Ethylene Oligomerization to α-olefins Using Cr(acac)₃, Bis(diphenylphosphino)isopropylamine (PNP) Ligand: Materials used for the oligomerization: Cr(acac)₃; Methylaluminoxane (MAO); toluene (Sigma Aldrich); Bis(diphenylphosphino)isopropylamine (PNP) ligand was synthesized in the laboratory and the method of preparation was explained above.

A solution of 30.0 mg of PNP ligand in 10 ml of toluene was added to a solution of 12.4 mg Cr(acac)₃ (0.033 mmol) in 10 mL toluene in a Schlenk vessel. The mixture was stirred for 5 minutes at ambient temperature and was then transferred to a 800 mL pressure reactor (autoclave) containing a mixture of toluene (80 mL) and MAO (methylaluminoxane, 9.9 mmol) at 60° C. The pressure reactor was charged with ethylene after which the reactor temperature was controlled at 65° C., while the ethylene pressure was maintained at 30 bar. The reaction was terminated after 60 minutes by discontinuing the ethylene feed to the reactor and cooling the reactor to below 10° C. After releasing the excess ethylene from the autoclave, the liquid contained in the autoclave was quenched with ethanol followed by 10% hydrochloric acid in water. A small sample of the organic layer was dried over anhydrous sodium sulfate and then analyzed by GC-FID. The remainder of the organic layer was filtered to isolate the solid products. These solid products were dried overnight in an oven at 100° C. and then weighed.

### Example 10

Ethylene Oligomerization to α-olefins at Atmospheric Pressure: Materials used for the oligomerization: Cr(acac)₃; modified methylaluminoxane (MMAO-12, Sigma Aldrich); toluene (Sigma Aldrich); Bis(diphenylphosphino)isopropylamine (PNP) ligand was synthesized in the laboratory and the method of preparation was explained above.

An ethylene tetramerization reaction was carried out in a 1 L round bottom flask under ambient temperature and pressure. A solution of 60 mg of PNP and 25 mg of Cr(acac)₃ in 40 mL toluene in a 1 L round bottom flask under highly inert atmosphere was stirred for 5 minutes at ambient temperature and then transferred to a flask containing a mixture 160 mL of toluene and 4.0 mL of MMAO-12 at room temperature. Initially the reactor was charged with hydrogen for 20 minutes, then switched to ethylene. The reaction was terminated after 24 hours by discontinuing the ethylene feed to the reactor. The liquid was quenched with ethanol followed by 10% hydrochloric acid in water. A small sample of the organic layer was dried over anhydrous sodium sulfate and then analyzed by GC-FID.

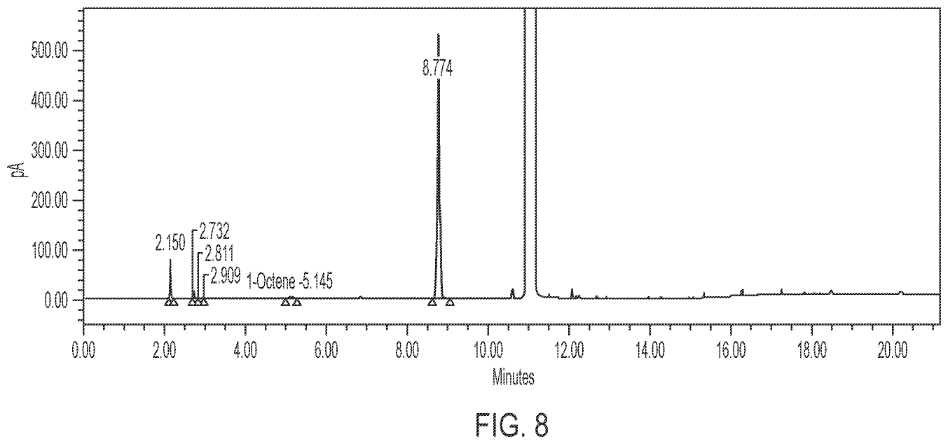

Results of the GC-FID analysis are reproduced in FIG. 8. Referring to FIG. 8, the 1-octene peak occurs at approximately 5 minutes of retention time, and reflects that a minimal amount of 1-octene was present in the analyzed sample.

### Example 11

Ethylene Oligomerization to α-olefins at 300 psi of Ethylene Pressure: Materials used for the oligomerization: Cr(acac)₃; modified methylaluminoxane (MMAO-12, Sigma Aldrich); toluene (Sigma Aldrich); Bis(diphenylphosphino)isopropylamine (PNP) ligand was synthesized in the laboratory and the method of preparation was explained above.

An ethylene tetramerization reaction was carried out in an 800 mL autoclave reactor under mild reaction conditions. A solution of 60 mg of PNP and 25 mg of Cr(acac)₃ in 40 mL toluene in a 100 mL round bottom flask under highly inert atmosphere was stirred for 5 minutes at ambient temperature and then transferred to a reactor (autoclave) containing a mixture of 160 mL toluene and 4.0 mL of MMAO-12 at 2-4° C. The reactor was pressurized to 300 psi with ethylene and reactor pressure and temperature was maintained at 300 psi and 2-4° C. The reaction was terminated after 7 hours by discontinuing the ethylene feed to the reactor. After releasing the excess ethylene from the autoclave, the liquid was quenched with ethanol followed by 10% hydrochloric acid in water. A small sample of the organic layer was dried over anhydrous sodium sulfate and then analyzed by GC-FID. The remainder of the organic layer was filtered to isolate the solid products. These solid products were dried overnight in an oven at 50° C. and then weighed.

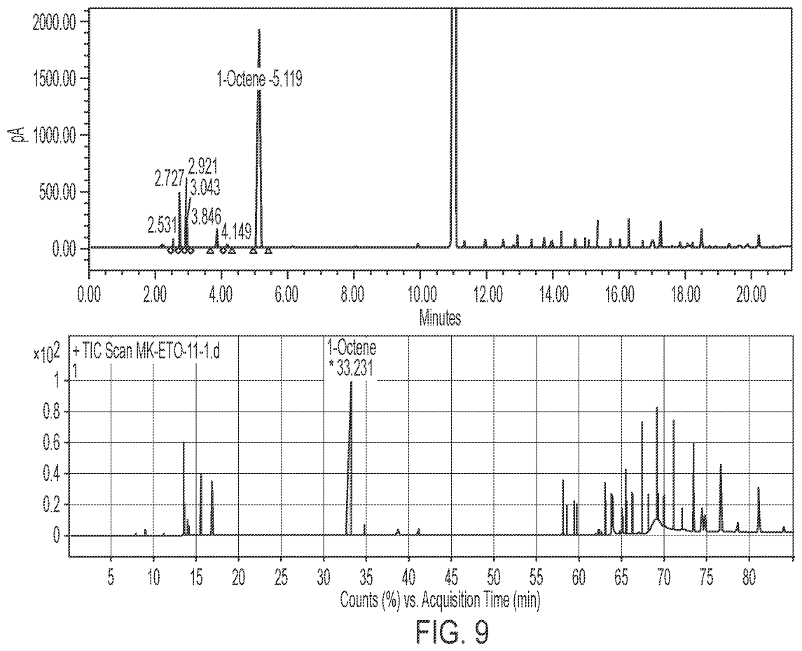

Results of the GC-FID and a mass spectral analysis on the same sample are depicted in FIG. 9. Referring to FIG. 9, both the GC-FID and mass spectroscopy plots indicate large peaks corresponding to 1-octene, indicating a significant concentration of 1-octene in the analyzed samples.

### Example 12

Ethylene Oligomerization to α-olefins at 300 psi of Ethylene, H₂ as Promoter: Materials used for the oligomerization: Cr(acac)₃; modified methylaluminoxane (MMAO-12, Sigma Aldrich); toluene (Sigma Aldrich); Bis(diphenylphosphino)isopropylamine (PNP) ligand was synthesized in the laboratory and the method of preparation was explained above.

An ethylene tetramerization reaction was carried out in an 800 mL autoclave reactor under mild reaction conditions. A solution of 60 mg of PNP and 25 mg of Cr(acac)₃ in 40 mL toluene in a 100 mL round bottom flask under highly inert atmosphere was stirred for 5 minutes at ambient temperature and then transferred to a reactor (autoclave) containing a mixture 160 mL toluene and 4.0 mL of MMAO-12 at 2-4° C. Initially the reactor was pressurized to 45 psi with hydrogen, then switched to ethylene and reactor pressure and temperature was maintained at 300 psi and 2-4° C. The reaction was terminated after 22 hours by discontinuing the ethylene feed to the reactor. After releasing the excess ethylene from the autoclave, the liquid was quenched with ethanol followed by 10% hydrochloric acid in water. A small sample of the organic layer was dried over anhydrous sodium sulfate and then analyzed by GC-FID. The remainder of the organic layer was filtered to isolate the solid products. These solid products were dried overnight in an oven at 50° C. and then weighed.

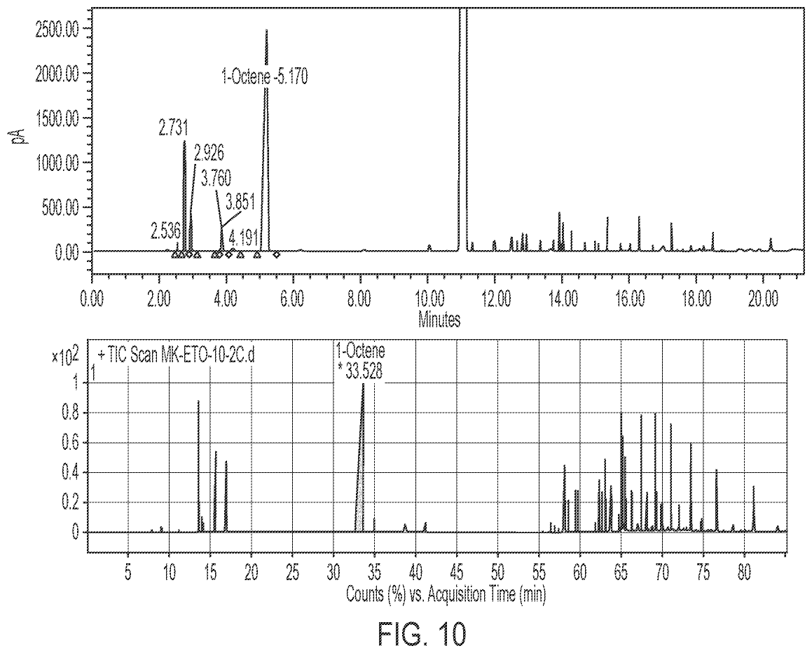

Results of the GC-FID and a mass spectral analysis on the same sample are depicted in FIG. 10. Referring to FIG. 10, both the GC-FID and mass spectroscopy plots indicate large peaks corresponding to 1-octene, indicating a significant concentration of 1-octene in the analyzed samples.

### Example 13

Ethylene Oligomerization to α-olefins at 300 psi of Ethylene, H₂ as Promoter: Materials used for the oligomerization: Cr(acac)₃; modified methylaluminoxane (MMAO-12, Sigma Aldrich); toluene (Sigma Aldrich); Bis(diphenylphosphino)isopropylamine (PNP) ligand was synthesized in the laboratory and the method of preparation was explained above.

An ethylene tetramerization reaction was carried out in an 800 mL autoclave reactor under mild reaction conditions. A solution of 60 mg of PNP and 25 mg of Cr(acac)₃ in 40 mL toluene in a 100 mL round bottom flask under highly inert atmosphere was stirred for 5 minutes at ambient temperature and then transferred to a reactor (autoclave) containing a mixture of 160 mL toluene and 4.0 mL of MMAO-12. Initially the reactor was pressurized to 40 psi with hydrogen, then switched to ethylene and reactor pressure and temperature was maintained at 300 psi and 19-20° C. The reaction was terminated after 4 hours by discontinuing the ethylene feed to the reactor. After releasing the excess ethylene from the autoclave, the liquid was quenched with ethanol followed by 10% hydrochloric acid in water. A small sample of the organic layer was dried over anhydrous sodium sulfate and then analyzed by GC-FID. The remainder of the organic layer was filtered to isolate the solid products. These solid products were dried overnight in an oven at 50° C. and then weighed.

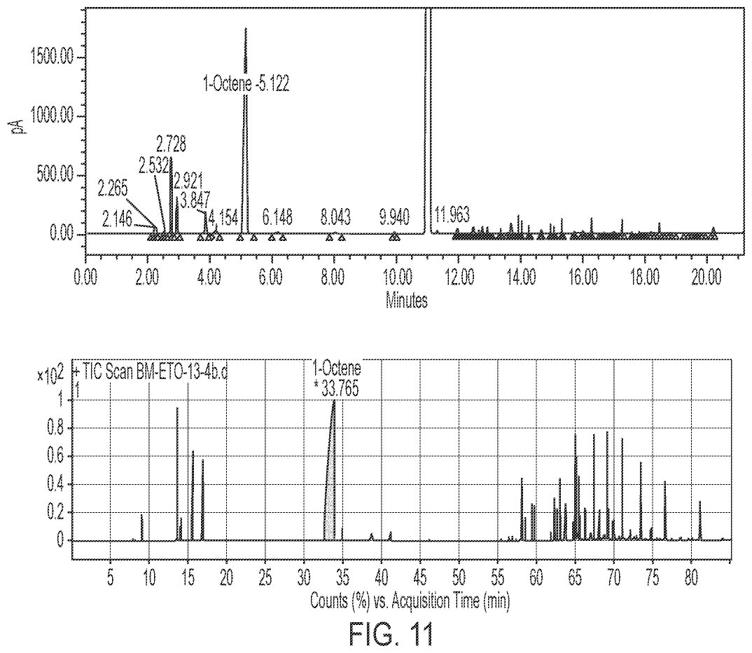

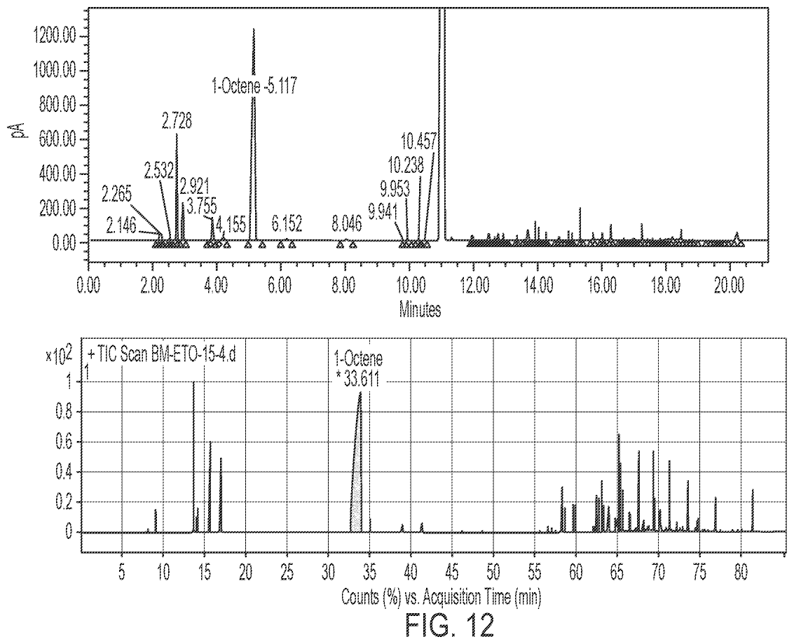

Results of the GC-FID and a mass spectral analysis on the same sample are depicted in FIG. 11. Referring to FIG. 11, both the GC-FID and mass spectroscopy plots indicate large peaks corresponding to 1-octene, indicating a significant concentration of 1-octene in the analyzed samples.

### Example 14

Ethylene Oligomerization to α-olefins at 200 psi of Ethylene, H₂ as Promoter: Materials used for the oligomerization: Cr(acac)₃; modified methylaluminoxane (MMAO-12, Sigma Aldrich); toluene (Sigma Aldrich); Bis(diphenylphosphino)isopropylamine (PNP) ligand was synthesized in the laboratory and the method of preparation was explained above.

An ethylene tetramerization reaction was carried out in an 800 mL autoclave reactor under mild reaction conditions. A solution of 60 mg of PNP and 25 mg of Cr(acac)₃ in 40 mL toluene in a 100 mL round bottom flask under highly inert atmosphere was stirred for 5 minutes at ambient temperature and then transferred to a reactor (autoclave) containing a mixture 160 mL of toluene and 4.0 mL of MMAO-12. Initially the reactor was pressurized to 45 psi with hydrogen, then switched to ethylene and reactor pressure and temperature was maintained at 200 psi and 19-20° C. The reaction was terminated after 4 hours by discontinuing the ethylene feed to the reactor. After releasing the excess ethylene from the autoclave, the liquid was quenched with ethanol followed by 10% hydrochloric acid in water. A small sample of the organic layer was dried over anhydrous sodium sulfate and then analyzed by GC-FID. The remainder of the organic layer was filtered to isolate the solid products. These solid products were dried overnight in an oven at 50° C. and then weighed.

Results of the GC-FID and a mass spectral analysis on the same sample are depicted in FIG. 12. Referring to FIG. 12, both the GC-FID and mass spectroscopy plots indicate large peaks corresponding to 1-octene, indicating a significant concentration of 1-octene in the analyzed samples.

Table 2 provides the concentration of 1-octene obtained for the experiments described in some of the examples above.

TABLE 2

| | | Concentration 1-octene |
|---|---|---|
| Experiment | Reaction Time (hr) | (g/L) |
| Example 10 | 24 | 0.03 |
| Example 11 | 7 | 38.4 |
| Example 12 | 6 | 24.6 |
| Example 12 | 22 | 104.5 |
| Example 13 | 2 | 113.5 |
| Example 13 | 4 | 178.2 |
| Example 14 | 2 | 63.0 |
| Example 14 | 4 | 128.6 |

### Example 15

Dihydroxylation of 1-octene to 1,2-octane diol: A 1 L round-bottomed flask equipped with magnetic stirring bar and overpressure valve NaIO₄ (21.4 g, 100 mmol) was stirred in 50 mL H₂O. Concentrated H₂SO₄ was added drop wise until all the solids were dissolved (about 4 mL) and the solution was cooled to 0° C. A 0.1 M aqueous solution of RuCl₃ (3.36 mL, 0.336 mmol) was added and the mixture was stirred until the color turned bright yellow. 200 mL of ethyl acetate was added and stirring was continued for 5 minutes. Acetonitrile (200 mL) was added and stirring was continued for an additional 5 minutes. The olefin (64 mmol) was added in one portion and the resulting slurry was stirred until all starting material was consumed (about 30-40 minutes). The mixture was poured onto 100 mL sat. NaHCO₃— and 100 mL sat. Na₂S₂O₃-solution. Phases were separated and the aqueous layer was extracted with ethyl acetate (3×50 mL). After drying the combined organic layer over Na₂SO₄ and evaporation of the solvent in vacuum the crude product was obtained. The crude product was purified by flash chromatography.

### Example 16

Synthesis of Ethylene-Vinyl Acetate (EVA) Copolymer: Materials used for EVA Synthesis: 2,2′-Azobis (isobutyronitrile), Tetrahydrofuran, Vinyl Acetate was purchased form Sigma-Aldrich.

EVA synthesized by free radical copolymerization of ethylene with vinyl acetate was performed in organic solvents under mild conditions. The reaction was initiated by 2,2′-Azobis (isobutyronitrile) [AIBN]. 100 mg of AIBN was dissolved under inert atmosphere in a mixture of the polymerization solvent tetrahydrofuran (THF) (60 mL) and vinyl acetate (40 mL). The mixture was introduced through cannula into a 300 mL stainless steel reactor (from Parr Instruments) equipped with safety valves and mechanical stirrer. Ethylene was introduced until the reactor pressure reached to 50 bar and same time reactor was heated to 70° C. under stirring rate of 400 rpm. After reaching the temperature, ethylene pressure was set to 75 bar throughout the reaction. After 5 hours of reaction time the reactor was slowly cooled down and degassed. The EVA copolymer was recovered by evaporation of solvent at room temperature.

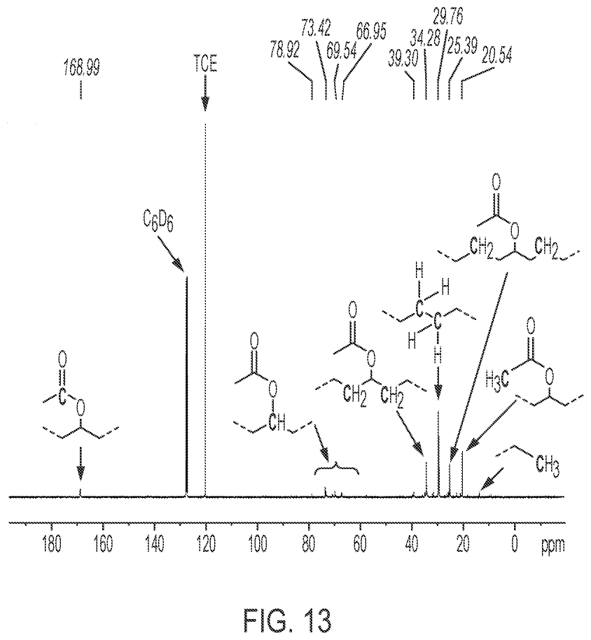

Results of a ¹³C NMR spectrum of the EVA copolymer (TCE—Tetrachloroethylene) obtained from Example 16 are depicted in FIG. 13. The solvent used was a 2:1 mixture of TCE and benzene-d6.

### Example 17

Synthesis of Phenol from Guaiacol: Materials used: Guaiacol (Sigma Aldrich), Carbon (CABOT, NORIT RX3 Extra), Ammonium molybdate tetrahydrate, (NH₄)₆Mo₇O₂₄.4H₂O) (Fluka Analytical), HNO₃ (VWR).

Carbon supported metal catalysts were prepared by incipient wetness impregnation method. Before impregnation, commercial activated carbon was treated with nitric acid by using refluxing method at 80° C. over 6 hours. After the acid treatment, carbon support was washed with DI water and dried at 110° C. overnight under vacuum.

1% Mo/C was prepared as follows. Oxidized carbon was used for the metal impregnation. Metal impregnation was performed using required amount of ammonium molybdate tetrahydrate precursor salt dissolved in DI water. After impregnation of metal on the support, the catalyst was kept at room temperature for 4 hours and then dried at 100° C. under vacuum. The resulting catalyst was calcined at 400° C. over 5 hours using Ar at 50 cc/min.

The reaction conditions for the demethoxylation of guaiacol to phenol were as follows. The catalyst volume was 12 cc (6.4 g). The reactor temperature was 300-400° C. at a reactor pressure of 400-600 psi. The hydrogen flow was 100 −3 ml/min and the liquid flowrate was 0.1-0.3 ml/min.

The catalytic activity measurements were performed in a fixed bed tubular reactor system with down flow mode. Before the reaction, the catalyst was reduced in situ in flowing H₂ at 100 cc/min for 1 hour at a temperature of 450° C. and pressure of 300 psi. After the reduction, the reactor was cooled down to the reaction temperature, the pressure was adjusted to the reaction pressure, and the guaiacol was fed into the reactor. The reaction products were condensed to 5° C. and liquid samples were collected from gas liquid separator within 2 hour intervals.

An 80-95% conversion of guaiacol was observed with a 40-70% observed selectivity to phenol and an 80-95% selectivity to phenolic compounds.

### Example 18

Synthesis of 2-Chloroethanol using Bio-Ethylene: Materials used: trichlorisocyanuric acid and acetone, obtained from Sigma Aldrich.

A solution of water/acetone (at a 1:5 volume ratio) was taken into a round bottom flask. The solution was saturated with ethylene at 300 cc/min for 20 minutes under constant stirring. A solution of 18% trichloroisocyanuric acid dissolved in water/acetone was added to the above saturated solution continuously. Samples were collected at 1 hour intervals to monitor the formation of 2-chloroethanol.

A concentration of 10-30% 2-chloroethanol was achieved.

### Example 19

Synthesis of Phenoxyethanol from Sodium Phenoxide: Materials used: sodium phenoxide trihydrate (Sigma Aldrich), 2-chloroethanol (Alfa-Aesar), sodium hydroxide (Alfa-Aesar), methylene chloride (Sigma Aldrich).

Phenoxyethanol was synthesized from a reaction of phenolate with 2-chloroethanol at a reaction temperature that is less than or equal to a boiling point of the reaction mixture to produce products that include the phenoxyethanol. Twenty grams of sodium phenolate trihydrate was added to 52 g of water and the mixture was stirred until dissolved in a round bottom flask. A solution of 9.45 g 2-chloroethanol in 7.8 g water was prepared separately. The aqueous solution of sodium phenolate was heated to 70° C. and the aqueous solution of 2-chloroethanol was added dropwise continuously during 1 hour time period while maintaining the temperature at 70° C. under constant stirring with refluxing condition. The reaction was performed for 6 hours, and the reaction mixture was thereafter allowed to cool to room temperature. The product was extracted with methylene chloride to form an organic phase. The organic phase was washed twice with a 5% aqueous solution of sodium hydroxide, and the solvent was distilled off. Phenoxyethanol was fractionally distilled under decreased pressure in an apparatus comprising a packed column, where a fraction with a boiling point within the range of 95° C. to 120° C. was collected. Fourteen grams of phenoxyethanol was obtained which is 80% w/w of the theoretical yield.

### Example 20

Purification of Ethylene Using Absorbents to Remove CO₂ and CO: Bio-ethylene production at high temperatures contains CO and CO₂ as impurities at elevated levels. Further polymerization and oligomerization reactions require ultra-pure ethylene. Exit gas flow of dehydration of ethanol was passed through the columns containing molecular sieves (5 A), molecular sieves (3 A), and Carbolime and Cu exchanged zeolite at room temperature.

### Example 21

Mass Production of 1,2-Octanediol from 1-Octene: FIG. 14 schematically depicts an exemplary mass production process for 1,2-octanediol (caprylyl glycol) from purified 1-octene. NaIO₄ in water and sulfuric acid may be fed into a reactor with RuCl₃, ethyl acetate, and acetonitrile. These reactants may then be fed into a batch reactor with purified 1-octene. The resultant stream may then be fed into another batch reactor with saturated NaHCO₃ and saturated Na₂SO₃. Following separation, the aqueous phase may be fed with additional ethyl acetate and distilled. The organic phase may be dried to remove solvent and obtain purified 1,2-octanediol (caprylyl glycol).

Those of skill in the art will appreciate that blends of bio-based and fossil fuel-derived 1-octene may be utilized in order to balance costs, sustainability, and environmental concerns, including GWP. Those of skill in the art will also appreciate from FIG. 14 that bio-based 1,2-octanediol (bio-based caprylyl glycol) having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

The following bench-scale synthesis was carried out. 21.4 g of NaIO₄ (100 mmol concentration) was introduced into a one-liter round bottom flask equipped with a magnetic stirring bar and overpressure valve and stirred in 50 ml water. Concentrated sulfuric acid was added dropwise until the solids were dissolved and the solution was cooled to 0° C. A 0.1 M aqueous solution of RuCl₃ (3.36 ml, 0.336 mmol) was added and the mixture was stirred until a bright yellow color was observed. 200 ml of ethyl acetate was added and stirring was continued for 5 minutes. Acetonitrile (200 ml) was added and stirring was continued for an additional 5 minutes. Olefil (64 mmol) was added in one portion and the resulting slurry was stirred until all starting material was consumed (30-40 minutes). The mixture was poured onto 100 ml saturated NaHCO₃ and 100 ml saturated Na₂SO₃ solution. The phases were separated, and the aqueous layer was extracted with ethyl acetate (3×50 ml). After drying the combined organic layer over Na₂SO₄ and evaporation of the solvent in vacuum the crude product was obtained. The crude product was then purified by flash chromatography to obtain 1,2-octanediol.

### Example 22

Mass Production of Phenoxyethanol from Guaiacol and Ethanol: FIG. 15 schematically depicts an exemplary mass production process for phenoxyethanol from guaiacol and ethanol. Hydrogen and guaiacol may be fed into a tubular reactor (fixed bed). Ethanol may be fed into a tubular reactor (fixed bed). Following gas-liquid separation of the ethanol, trichloroisocyanuric acid may be added to a batch reactor to obtain chloroethanol, which is separated from by-products. Following gas-liquid separation of the reactant stream from the hydrogen and guaiacol reaction, phenol may be obtained. The chloroethanol and phenol may then be fed into a batch reactor. Phenoxyethanol may be obtained following distillation of the reactant stream.

The raw materials in this process may be bio-based and have a high modern carbon content and pMC. Therefore, the phenoxyethanol that is produced may also be bio-based and have a high modern carbon content and pMC. Those of skill in the art will appreciate that blends of bio-based and fossil fuel-derived guaiacol and ethanol may be utilized in order to balance costs, sustainability, and environmental concerns, including GWP. Those of skill in the art will also appreciate from FIG. 15 that bio-based phenoxyethanol having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

### Example 23

Mass Production of Ethylene from Ethanol: FIG. 16 schematically depicts another exemplary mass production process for ethylene from ethanol. Ethanol may be fed into a preheater and subsequently heated to 400° C. Nitrogen may also be heated to 400° C. and combined with the ethanol stream in a reactor. The reactant stream may be cooled and separated by distillation to obtain ethylene.

The raw materials in this process may be bio-based and have a high modern carbon content and pMC. Therefore, the ethylene that is produced may also be bio-based and have a high modern carbon content and pMC. Those of skill in the art will appreciate that blends of bio-based and fossil fuel-derived ethanol may be utilized in order to balance costs, sustainability, and environmental concerns, including GWP. Those of skill in the art will also appreciate from FIG. 16 that bio-based ethylene having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

The following bench-scale synthesis was carried out. A reactor was loaded with 8 grams of 1% La/γ Al₂O₃ catalyst. The catalyst was pre-activated at 400° C. under 100 ml/min nitrogen for 2 hours. Ethanol dehydration was carried out in a fixed bed reactor at a temperature of 400° C. and ambient pressure using a nitrogen flow rate of 30-50 ml/min. The preheated bio-based ethanol was injected into the catalyst bed with a liquid flow rate of 0.3-0.5 ml/min.

### Example 24

Mass Ethylene Oligomerization to Alpha-Olefins: FIG. 17 schematically depicts an exemplary mass production process from ethylene. Cr(acac)₃, PNP, MMAO-12, and toluene may be added to a mixing vessel and fed into a pressure reactor. Ethylene and nitrogen may be fed into the pressure reactor and the pressure reactor at an initial pressure at 50 psi of hydrogen. The reaction may be carried out at a temperature of 0-20 C and at a pressure of 100-300 psi. Following separation, 1-octene in toluene and polymers may be obtained.

The raw materials in this process may be bio-based and have a high modern carbon content and pMC. Therefore, the 1-octene and polymers that are produced may also be bio-based and have a high modern carbon content and pMC. Those of skill in the art will appreciate that blends of bio-based and fossil fuel-derived ethylene may be utilized in order to balance costs, sustainability, and environmental concerns, including GWP. Those of skill in the art will also appreciate from FIG. 17 that bio-based 1-octene and polymers having any desired pMC content can be obtained by utilizing mixtures of bio-based materials and fossil fuel-based materials, from low levels, such as 1 to 2 pMC to medium levels, such as 40 to 60 pMC, to higher levels, such as 80 to 99 pMC, or nearly 100 pMC.

The following bench-scale synthesis was carried out. Ethylene tetramerization was carried out in an 800 ml autoclave reactor under mild reaction conditions. A solution of 60 mg PNP and 25 mg Cr(acac)₃ in 40 ml toluene was mixed in a 100 ml round-bottom flask under highly inert atmosphere. The mixture was stirred for 5 minutes at ambient temperature and then transferred to an autoclave reactor containing a mixture of 160 ml toluene and 4.0 ml of MMAO-12. Initially, the reactor was pressurized to 40 psi with hydrogen, then switched to ethylene and the reactor pressure was maintained at 300 psi at a temperature of 19-20° C. The reaction was terminated after 4 hours by discontinuing the ethylene feed to the reactor. After releasing the excess ethylene from the autoclave, the liquid was quenched with ethanol followed by 10% hydrochloric acid in water. A small sample of the organic layer was dried over anhydrous sodium sulfate and then analyzed by GC-FID. The remainder of the organic layer was filtered to isolate the solid products.

Other than in the examples herein, or unless otherwise expressly specified, all of the numerical ranges, amounts, values, and percentages, such as those for amounts of materials, elemental contents, times and temperatures of reaction, ratios of amounts, and others, may be read as if prefaced by the word “about” even though the term “about” may not expressly appear with the value, amount, or range. In addition, when numerical ranges are set forth herein, these ranges are inclusive of the recited range end points (i.e., end points may be used). When percentages by weight are used herein, the numerical values reported are relative to the total weight. Furthermore, any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein, but which conflicts with the statements or other disclosure material set forth herein, will only be incorporated to the extent that no conflict arises between that incorporated material and the disclosure set forth herein. To the extent necessary, the disclosure explicitly set forth herein supersedes any conflicting material incorporated herein by reference.

While this invention has been particularly shown and described with references to preferred embodiments thereof, in light of the present disclosure it will be understood by persons skilled in the art that various changes in form and details may be made therein without departing from the scope of the invention encompassed by the appended claims.

All references, patents, and publications disclosed herein are hereby incorporated by reference in their entireties.

1. A composition comprising bio-based ethylene, wherein the percent modern carbon (pMC) of the composition is at least about 50 pMC.

2. The composition of claim 1, wherein the percent modern carbon (pMC) of the composition is at least about 100 pMC.

3. A composition comprising bio-based polyethylene, wherein the percent modern carbon (pMC) of the composition is at least about 50 pMC.

4. The composition of claim 3, wherein the percent modern carbon (pMC) of the composition is at least about 100 pMC.

5. The composition of claim 1, wherein the percent bio-based carbon of the composition is at least about 50%.

6. The composition of claim 1, wherein the percent bio-based carbon of the composition is at least 95%.

7. The composition of claim 3, wherein the percent bio-based carbon of the composition is at least about 50%.

8. The composition of claim 3, wherein the percent bio-based carbon of the composition is at least 95%.

9-172. (canceled)
